(19) United States
(12) Patent Application Publication
(10) Pub. No.: US 2024/0366569 A1
(43) Pub. Date: Nov. 7, 2024

(54) COMBINATIONS FOR TREATING CANCER

(71) Applicant: Purdue Pharmaceutical Products L.P., Stamford, CT (US)

(72) Inventors: Markus Jörger, St. Gallen (CH); Lukas Flatz, St. Gallen (CH)

(21) Appl. No.: 18/654,296

(22) Filed: May 3, 2024

Related U.S. Application Data
(60) Provisional application No. 63/464,343, filed on May 5, 2023.

(51) Int. Cl. A61K 31/4184; A61K 39/00; A61P 35/00

(52) U.S. Cl. CPC A61K 31/4184; A61P 35/00; A61K 2039/507

(57) ABSTRACT

The invention relates to combinations, pharmaceutical compositions and kits comprising Tinostamustine, and an immune checkpoint inhibitor. The combinations, pharmaceutical compositions and kits are effective for use in the treatment of cancer, particularly advanced cancer.

### REFERENCE TO RELATED APPLICATIONS

This application claims priority to U.S. Provisional Application No. 63/464,343, filed on May 5, 2023. The entire contents of the foregoing application, including all drawings, are expressly incorporated herein by reference.

### FIELD OF THE INVENTION

The present invention relates to a combination comprising Tinostamustine, or a pharmaceutically acceptable salt thereof, and an immune checkpoint inhibitor. The present invention further relates to medical uses of said combination, and in particular the combinations described herein for use in the treatment of cancer.

### BACKGROUND

The manipulation of the immune system by blocking ligands and receptors that act as regulators of the immune response (so-called immune checkpoints) has become an important strategy in the treatment of cancer in recent years. Therapies that block ligands and receptors that act as regulators of the immune response may be referred to in the art as “immune checkpoint inhibitors”. To date, several immune checkpoint inhibitors have been approved for clinical use in a variety of cancers. Examples of immune checkpoint inhibitors include cytotoxic T lymphocyte associated antigen 4 (CTLA-4) inhibitors and Programmed cell death protein 1 (PD-1) inhibitors, as well as inhibitors of its ligand (PD-L1).

Programmed cell death protein-1 (PD-1, CD279), a 55 kD type I transmembrane protein, is a member of the CD28 family of T-cell costimulatory receptors that also includes CD28, CTLA-4, ICOS, and BTLA. PD-1 contains an intracellular membrane proximal immunoreceptor tyrosine inhibitory motif (ITIM) and a membrane distal immunoreceptor tyrosine-based switch motif (ITSM). PD-1 consists of 4 polypeptide chains, which include 2 identical heavy chains and 2 identical light chains. PD-1 is a negative regulatory molecule primarily expressed on activated T-cells, B-cells and myeloid cells (Nishimura, H. and T. Honjo, PD-1: an inhibitory immunoreceptor involved in peripheral tolerance. Trends Immunol, 2001. 22(5): p. 265-8). PD-1 delivers a negative signal by the recruitment of a protein tyrosine phosphatase SHP-2 to the phosphorylated tyrosine residue in the ITSM in its cytoplasmic region (Sheppard, K. A., et al., PD-1 inhibits T-cell receptor induced phosphorylation of the ZAP70/CD3zeta signalosome and downstream signaling to PKCtheta. FEBS Lett, 2004. 574(1-3): p. 37-41).

Two ligands specific for PD-1 have been identified, namely PD-L1 (B7-H1/CD274) and PD-L2 (B7-DC/CD273). PD-L1 and PD-L2 have been shown to down-regulate T-cell activation upon binding to PD-1 in both murine and human systems (Latchman, Y., et al., PD-L2 is a second ligand for PD-1 and inhibits T cell activation. Nat Immunol, 2001. 2(3): p. 261-8; Carter, L., et al., PD-1:PD-L inhibitory pathway affects both CD4(+) and CD8(+) T cells and is overcome by IL-2. Eur J Immunol, 2002. 32(3): p. 634-43).

Nivolumab, is a human monoclonal antibody (immunoglobulin G4 [IgG4]-S228P) that targets the Programmed death-1 (PD-1). PD-1 blockade by Nivolumab is considered a promising immunotherapy, and clinical approval has been received for Nivolumab in several cancer types including advanced NSCLC, melanoma, renal-cell cancer, MSI-high colorectal cancer, squamous-cell cancer of the head and neck and Hodgkin's disease. Nivolumab has shown improved overall survival in patients with advanced melanoma compared to dacarbazine chemotherapy when given in first-line according to the CheckMate 066 clinical trial (Robert, C., et al., Nivolumab in previously untreated melanoma without BRAF mutation. N Engl J Med, 2015. 372(4): p. 320-30). In CheckMate 066, overall survival was improved by 58% (HR=0.42, p<0.001), while the objective response rate was improved from 13.9% with dacarbazine to 40.0% with Nivolumab, and progression-free survival was improved by 57% from 2.2 to 5.1 months (HR=0.43, p<0.001).

Nivolumab has also received approval as a combination therapy with the anti-CTLA-4 monoclonal antibody Ipilimumab in patients with advanced melanoma based on the results of the CheckMate 067 study that showed improved median overall survival with the combination versus Nivolumab monotherapy (37.6 months versus not reached, HR=0.85, (95% Cl, 0.68 to 1.07)) (Wolchok, J. D., et al., Overall Survival with Combined Nivolumab and Ipilimumab in Advanced Melanoma. N Engl J Med, 2017. 377(14): p. 1345-1356). However, treatment of patients with a combination of Nivolumab and Ipilimumab has led to significant treatment-related adverse events of grade 3 or 4 in 59% of patients. Consequently, the administration of a combination of Nivolumab and Ipilimumab is restricted to younger patients with a higher performance score.

These data suggest that the current standard of care comprising either an immune checkpoint inhibitor as a monotherapy, or an immune checkpoint inhibitor in combination with another immune checkpoint inhibitor, can lead to adverse patient outcomes. There is therefore a need for improved immunotherapies for the treatment of cancer.

Melanoma is particularly challenging type of cancer to treat. Whilst melanoma represents only 1% of all malignancies, treatment is a challenge due to its high metastatic potential and mortality. Over recent decades, the number of cases of melanoma has increased dramatically, faster than any other type of cancer. While patients with early diagnosis have 5-year survival rates around 90%, this number decreases to 10% in patients with advanced melanoma, with a median survival of 6 to 12 months. Advanced melanoma has therefore traditionally been associated with limited therapeutic options and poor prognosis. Historically, median survival was in the range between 6 and 9 months but in recent years, the emergence of new therapeutic agents (e.g. immune checkpoint inhibitors) has improved treatment of advanced melanoma. Objective responses with immune checkpoint inhibitors can be seen in more than 50% of advanced melanoma patients, and a 25-35% probability of a patient being alive following treatment has transitioned from 12 months to 48-60 months (Hodi F S, K. H., Sznol M, Carvajal R, Lawrence D, Atkins M, et al., Durable, long-term survival in previously treated patients with advanced melanoma (MEL) who received nivolumab (NIVO) monotherapy in a phase I trial. Cancer Res., 2016. 76). Nevertheless, approximately 9,000 patients still die of melanoma annually in the U.S., highlighting the need for more effective treatments.

WO 2010/085377 discloses Tinostamustine (also referred to as EDO-S101), which is a first-in-class alkylating deacetylase inhibiting (HDACi) molecule. Tinostamustine has shown anti-cancer activity in in vitro and in vivo models, and is the subject of clinical trials for the treatment of glioblastoma (NCT05432375, NCT03452930), relapsed/refractory hematologic malignancies (NCT02576496), and solid tumours (NCT03345485).

It is an object of the present invention to address one or more of the aforementioned problems.

### SUMMARY OF INVENTION

The present invention relates to the surprising and unexpected discovery that the efficacy of immune checkpoint inhibitors in the treatment of cancer can be increased, when the immune checkpoint inhibitor is administered in combination with the alkylating-HDACi molecule, Tinostamustine. Without wishing to be bound by theory, it is thought that Tinostamustine improves the efficacy of the immune checkpoint inhibitor, thus leading to improved overall progression free survival, and improved overall survival, as demonstrated in the clinical data described herein. The clinical data described herein demonstrates that compared to the current standard of care for advanced melanoma, the combination according to the present invention increases median progression free survival (mPFS) and median overall survival (mOS) by several months. This represents a significant increase in survival for patients for whom surgery is not an option, and other courses of treatment including chemotherapy and radiotherapy have been deemed to be, or likely to be, ineffective. Given immune checkpoint inhibitors are used in the treatment of a wide range of cancers in addition to advanced melanoma, it is envisaged that the unexpected discovery that the combination of the present invention is effective in the treatment of advanced cancer, will have broad applicability in the treatment of cancer more generally.

Further still, it has been found that the combinations as described herein avoid adverse events associated with prior art combinations for treatment of cancer comprising two or more immune checkpoint inhibitors (e.g. a combination of Nivolumab and Ipilimumab). The present invention is therefore not only directed to combinations which improve patient prognosis (as determined by mPFS and mOS), but additionally mitigates adverse side effects associated with immunotherapy.

According to a first aspect of the present invention, the present disclosure provides a combination comprising Tinostamustine, or a pharmaceutically acceptable salt thereof, and an immune checkpoint inhibitor.

In some embodiments, the immune checkpoint inhibitor is a Programmed cell death protein-1 (PD-1) inhibitor. In certain embodiments, the immune checkpoint inhibitor is an anti PD-1 antibody. In some embodiments, the immune checkpoint inhibitor is selected from Pembrolizumab, Nivolumab, Dostarlimab, Retifanlimab and Cemiplimab. In one embodiment, the immune checkpoint inhibitor is Nivolumab.

In some embodiments, the immune checkpoint inhibitor is a Programmed cell death protein ligand 1 (PD-L1) inhibitor and/or Programmed cell death protein ligand 2 (PD-L2) inhibitor. In some embodiments, the immune checkpoint inhibitor is a PD-L1 inhibitor. In some embodiments, the immune checkpoint inhibitor is an anti PD-L1 antibody. In some embodiments, the immune checkpoint inhibitor is selected from Atezolizumab, Avelumab, and Durvalumab.

In some embodiments, the combination further comprises one or more further immune checkpoint inhibitors. In some embodiments, the one or more further immune checkpoint inhibitors may comprise a cytotoxic T lymphocyte associated antigen 4 (CTLA-4) inhibitor. In some embodiments, the CTLA-4 inhibitor is Ipilimumab. In some embodiments, the CTLA-4 inhibitor is Tremelimumab.

According to a second aspect of the present invention, the present disclosure provides a pharmaceutical composition comprising a combination according to the first aspect of the present invention, and a pharmaceutically acceptable diluent or carrier.

According to a third aspect of the present invention, the present disclosure provides a kit comprising a combination according to the first aspect of the present invention or a pharmaceutical composition according to the second aspect of the invention, and optionally, instructions for treating a patient.

In some embodiments, the combination according to the first aspect of the present invention, pharmaceutical composition according to the second aspect of the present invention, or kit according to the third aspect of the present invention, is provided for use in therapy (e.g. for use as a medicament).

According to a fourth aspect of the present invention, the present disclosure provides a method of treating cancer, comprising administering to a subject in need thereof a combination according to the first aspect of the present invention, pharmaceutical composition according to the second aspect of the present invention, or kit according to the third aspect of the present invention. In some embodiments, the cancer is selected from melanoma, small-cell lung cancer, non-small cell lung cancer, mesothelioma (e.g. malignant pleural mesothelioma), renal cell carcinoma, head and neck cancer, urothelial carcinoma, colorectal cancer, oesophageal squamous cell carcinoma, hepatocellular carcinoma, gastric cancer, oesophageal cancer, oesophageal adenocarcinoma, gastroesophageal junction cancer, Microsatellite Instability-High or Mismatch Repair Deficient Cancer, Primary Mediastinal Large B-Cell Lymphoma (PMBCL), Merkel Cell Carcinoma (MCC), Endometrial Carcinoma, Tumour Mutational Burden-High (TMB-H) Cancer, Cutaneous Squamous Cell Carcinoma (cSCC), soft tissue sarcoma, bone sarcoma, ovarian cancer, triple-negative breast cancer, brain cancer (e.g. glioblastoma), multiple myeloma, Hodgkin lymphoma and non-Hodgkin lymphoma. In some embodiments, the cancer is melanoma, optionally advanced melanoma. In some embodiments, the cancer is relapsed and/or refractory.

In some embodiments, Tinostamustine, or a pharmaceutically acceptable salt thereof, is administered to a patient in need thereof as an adjuvant and/or neoadjuvant. In one embodiment, Tinostamustine is administered to a patient in need thereof as a neoadjuvant.

In some embodiments, Tinostamustine, or a pharmaceutically acceptable salt thereof, is administered to the patient at a dosage range of from 10 to 50 mg/m², from 20 to 40 mg/m², or 25 to 35 mg/m² body surface area. One embodiment provides that the dosage is 30 mg/m² body surface area.

In some embodiments, Tinostamustine, or a pharmaceutically acceptable salt thereof, and the immune checkpoint inhibitor, are administered to the patient sequentially, concurrently or separately. In some embodiments, Tinostamustine, or a pharmaceutically acceptable salt thereof, and the immune checkpoint inhibitor are administered separately, optionally wherein the immune checkpoint is administered to the patient in need thereof first, followed by administration of Tinostamustine. In some embodiments, Tinostamustine or a pharmaceutically acceptable salt thereof is administered to the patient in need thereof from 20 to 120 minutes, from 30 to 90 minutes, or from 50 to 70 minutes (e.g. 60 minutes), after administration of the immune checkpoint inhibitor to the patient. In some embodiments, Tinostamustine or a pharmaceutically acceptable salt thereof is administered to the patient in need thereof at least 30 minutes after administration of the immune checkpoint inhibitor to the patient.

In some embodiments, for a first cycle of treatment Tinostamustine, or a pharmaceutically acceptable salt thereof, is administered to the patient in need thereof without the checkpoint inhibitor, and for all subsequent cycles (e.g., second, third, fourth cycles etc.), Tinostamustine and or a pharmaceutically acceptable salt thereof, and the immune checkpoint inhibitor, are administered to the patient sequentially, concurrently or separately, preferably separately.

According to a fifth aspect of the present invention, the present disclosure provides Tinostamustine, or a pharmaceutically acceptable salt thereof, for use in the treatment of cancer, wherein Tinostamustine is used in combination with an immune checkpoint inhibitor. The cancer and/or said treatment may be as defined in accordance with the embodiments of the fourth aspect of the invention.

According to a sixth aspect of the present invention, the present disclosure provides an immune checkpoint inhibitor for use in the treatment of cancer, wherein the immune checkpoint inhibitor is used in combination with Tinostamustine, or a pharmaceutically acceptable salt thereof. The cancer and/or said treatment may be as defined in accordance with embodiments of the invention.

According to a seventh aspect of the present invention, the present disclosure provides a method of treating cancer, the method comprising administering to a patient in need thereof a therapeutically effective amount of Tinostamustine, or a pharmaceutically acceptable salt thereof, in combination with a therapeutically effective amount of an immune checkpoint inhibitor. The cancer and/or said treatment may be as defined in accordance with the embodiments of the invention.

According to an eighth aspect of the present invention, the present disclosure provides use of a combination comprising Tinostamustine, or a pharmaceutically acceptable salt thereof, and an immune checkpoint inhibitor in the treatment of cancer. The cancer and/or said treatment may be as defined in accordance with the embodiments of the invention.

According to a ninth aspect of the present invention, the present disclosure provides use of Tinostamustine, or a pharmaceutically acceptable salt thereof, in the manufacture of a medicament for use in the treatment of cancer, wherein in said treatment Tinostamustine, or a pharmaceutically acceptable salt thereof, is used in treatment in combination with an immune checkpoint inhibitor. The cancer and/or said treatment may be as defined in accordance with the embodiments of the fourth aspect of the invention.

According to a tenth aspect of the present invention, the present disclosure provides use of an immune checkpoint inhibitor in the manufacture of a medicament for use in the treatment of cancer, wherein in said treatment the immune checkpoint inhibitor is used in treatment in combination with Tinostamustine, or a pharmaceutically acceptable salt thereof. The cancer and/or said treatment may be as defined in accordance with the embodiments of the invention.

According to an eleventh aspect of the present invention, the present disclosure provides a method of treating melanoma, where the method comprises administering to a subject in need thereof Tinostamustine, or a pharmaceutically acceptable salt thereof, optionally wherein the melanoma is advanced melanoma. In certain embodiments, the melanoma is relapsed and/or refractory.

According to a twelfth aspect of the present invention, the present disclosure provides Tinostamustine, or a pharmaceutically acceptable salt thereof, for use in the treatment of colorectal cancer, optionally wherein the colorectal cancer is a carcinoma. Certain embodiments provide that the carcinoma is an adenocarcinoma.

### BRIEF DESCRIPTION OF FIGURES

FIG. 1 illustrates progression free survival (PFS) for patients treated with a combination of Tinostamustine and Nivolumab, in accordance with the examples described herein, and a comparison of median progression free survival (mPFS) vs Standard of Care Reference (Zimmer et al). “mPFS All” refers to mPFS calculated for all patients recruited to the study, as described in the examples herein. “mPFS Responders” refers to mPFS calculated for patients who were deemed to be responsive to treatment, as described in the examples herein. “mPFS ipi/nivo (Zimmer)” refers to the reference mPFS value for patients treated with a combination of Ipilimumab and Nivolumab as provided by Zimmer et al as described in the examples herein. The patient reference number corresponds to the numeral given to each patient in Table 1 in the examples herein.

FIG. 2 illustrates overall survival (OS) for patients treated with a combination of Tinostamustine and Nivolumab, in accordance with the examples described herein, and a comparison of median overall survival (mOS) vs Standard of Care Reference (Baron et al). “mOS All” refers to mOS calculated for all patients recruited to the study, as described in the examples herein. “mOS Responders” refers to mOS calculated for patients who were deemed to be responsive to treatment, as described in the examples herein. “mOS ipi/nivo (Baron)” refers to the reference mOS value for patients treated with a combination of Ipilimumab and Nivolumab as provided by Baron et alas described in the examples herein. The patient reference number corresponds to the numeral given to each patient in Table 1 in the examples herein.

FIG. 3 shows that Tinostamustine as single agent or Tinostamustine as a combination treatment with an anti-PD1 antibody in the MC38 colorectal cancer model is well-tolerated and leads to weight gain. Mean body weights of mice in different groups during treatment in model MC38.

FIG. 4 shows that Tinostamustine as a combination treatment with an anti-PD1 antibody in a MC38 colorectal cancer model significantly reduces tumour burden compared to tinostamustine as a single agent and to the control. P-value, Ns=non-significant P>0.05; ***: P<0.001.

FIG. 5 shows Tinostamustine as single agent or combination treatment with an anti-PD1 antibody has anti-tumour effect in the treatment of MC38. a. Mean±SEM (mice number); b. TGI %=[1-T/C]×100%; RTV_TGI %=[1-RTV_T/RTV_C]×100% c. Bartlett's test was performed to test homogeneity of variance and normality, p-value<0.05; then Kruskal-Wallis test was performed, p-value<0.05; then Conover's non-parametric many-to-one comparison test was performed for each treatment against one control. ns: no-significant; ***: P<0.001.

FIG. 6 shows Tinostamustine as single agent or combination treatment with an anti-PD1 antibody increases survival of mice with colorectal cancer (MC38 Model). Since more than 50% of the mice were alive at the end of the study from Group 2 to Group 6, the median survival time was simply not defined in these groups. Survival time was assessed by time to the mice were found dead or the mice were sacrificed.

### DEFINITIONS

As used herein, the term “immune checkpoint inhibitor”, unless otherwise indicated, means drug molecules that are capable of binding to an immune checkpoint receptor on the surface of an immune cell to block said receptor and prevent interaction of said receptor with an endogenous ligand(s). Alternatively, the term “immune checkpoint inhibitor” may mean drug molecules capable of binding to a ligand(s) for an immune checkpoint receptor, to block said ligand from interacting with the immune checkpoint receptor. The immune checkpoint receptor is a protein, typically presented on the surface of an immune cell (e.g., T-cells) that are involved in checkpoint regulation of the immune response. The ligand for the immune checkpoint receptor is also a protein, and may be present, for instance, on the surface of a cancer cell.

More particularly, immune checkpoint inhibitors as described may include drugs that block or inhibit immune checkpoint receptors, optionally wherein immune checkpoint receptors may be selected from one or more of adenosine A2A receptor (A2AR/ADORA2A), adenosine A2B receptor (A2BR/ADORA2B), B7 Homolog 3 (CD276/B7-H3), V-set domain-containing T-cell activation inhibitor 1 (VTCN1/B7-H4), B- and T-lymphocyte attenuator (CD272/BTLA), Cytotoxic T-Lymphocyte-Associated protein 4 (CD152/CTLA-4), Indoleamine-pyrrole 2,3-dioxygenase (IDO), Killer-cell immunoglobulin-like receptors (KIR), Lymphocyte-activation gene 3 (LAG-3), NADPH oxidase 2 (NOX2/cytochrome b(558) subunit beta/Cytochrome b-245 heavy chain), Programmed cell death protein 1 (PD-1), T-cell immunoglobulin and mucin-domain containing-3 (TIM-3/Hepatitis A virus cellular receptor 2 [HAVCR2]), V-domain Ig suppressor of T cell activation (VISTA), sialic acid-binding Ig-like lectin 7 (SIGLEC7), and optionally ligands thereof.

In some embodiments, immune checkpoint inhibitors as described herein include drugs that block or inhibit immune checkpoint receptors selected from Programmed cell death protein 1 (PD-1) and Cytotoxic T-Lymphocyte-Associated protein 4 (CD152/CTLA-4), and optionally ligands thereof.

In some embodiments, immune checkpoint inhibitors as described herein include drugs that block or inhibit the immune checkpoint receptor Programmed cell death protein 1 (PD-1). In some embodiments, immune checkpoint inhibitors as described herein include drugs that block or inhibit ligands of the immune checkpoint receptor Programmed cell death protein 1 (PD-1), selected from Programmed cell death protein ligand 1 (PD-1) and Programmed cell death protein ligand 2 (PD-2).

In some embodiments, immune checkpoint inhibitors as described herein include monoclonal antibodies which target and bind to an immune checkpoint receptor selected from Programmed cell death protein 1 (PD-1) and Cytotoxic T-lymphocyte-Associated Protein 4 (CTLA-4) (Kim S K, Cho S W. The Evasion Mechanisms of Cancer Immunity and Drug Intervention in the Tumor Microenvironment. Front Pharmacol. 2022 May 24; 13:868695. doi: 10.3389/fphar.2022.868695. PMID: 35685630; PMCID: PMC9171538).

In some embodiments, immune checkpoint inhibitors as described herein include monoclonal antibodies which target and bind to Programmed cell death protein 1 (PD-1). In some embodiments, immune checkpoint inhibitors as described herein include monoclonal antibodies which target and bind to ligands of Programmed cell death protein 1 (PD-1), selected from Programmed cell death protein ligand 1 (PD-1) and Programmed cell death protein ligand 2 (PD-2).

As used herein, the term “Tinostamustine” refers to a compound having the following structural formula:

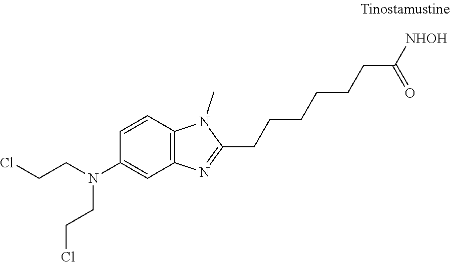

“Pharmaceutically acceptable salt” means a salt of the compounds of the present invention (e.g. Tinostamustine) which are pharmaceutically acceptable, and which may also possess the desired pharmacological activity. Such salts include acid addition salts formed with inorganic acids, or with organic acids. Pharmaceutically acceptable salts also include base addition salts which may be formed when acidic protons present are capable of reacting with inorganic or organic bases. Generally, such salts are, for example, prepared by reacting the free acid or base forms of these compounds with a stoichiometric amount of the appropriate base or acid in water or in an organic solvent or in a mixture of the two. Non-aqueous media like ether, ethyl acetate, ethanol, isopropanol or acetonitrile can be used. Examples of the acid addition salts include mineral acid addition salts such as, for example, hydrochloride, hydrobromide, hydroiodide, sulfate, bisulfate, sulfamate, nitrate, phosphate, and organic acid addition salts such as, for example, acetate, trifluoroacetate, maleate, fumarate, citrate, oxalate, succinate, tartrate, salicylate, tosylate, lactate, naphthalenesulphonae, malate, mandelate, methanesulfonate and p-toluenesulfonate. Examples of the alkali addition salts include inorganic salts such as, for example, sodium, potassium, calcium and ammonium salts, and organic alkali salts such as, for example, ethylenediamine, ethanolamine, N,N-dialkylenethanolamine, triethanolamine and basic amino acid salts.

As used herein, the term ‘antibody’, unless otherwise indicated refers to any immunoglobulin, such as a full-length immunoglobulin. Preferably the term covers monoclonal antibodies, polyclonal antibodies, multispecific antibodies, such as bispecific antibodies, and antibody fragments thereof, so long as they exhibit the desired biological activity. Antibodies may be derived from any species, but preferably are of mouse, human or rabbit origin. Alternatively, the antibodies, preferably monoclonal antibodies, may be humanised, chimeric or antibody fragments thereof. The term ‘chimeric antibodies’ may also include “primatised” antibodies comprising variable domain antigen-binding sequences derived from a non-human primate (e.g., Old World Monkey, Ape etc) and human constant region sequences. The immunoglobulins can also be of any type (e.g. IgG, IgE, IgM, IgD, and IgA), class (e.g., IgGI, IgG2, IgG3, IgG4, IgA1 and IgA2) or subclass of immunoglobulin molecule.

As used herein, the term ‘monoclonal antibody’, unless otherwise indicated, refers to a substantially homogenous population of antibody molecules (i.e. the individual antibodies comprising the population are identical except for possible naturally occurring mutations that may be present in minor amounts), produced by a single clone of B lineage cells, often a hybridoma. Importantly, each monoclonal antibody has the same antigenic specificity—i.e. it is directed against a single determinant on the antigen.

As used herein, the term “treating”, unless otherwise indicated, means reversing, attenuating, alleviating or inhibiting the progress of the disease or condition to which such term applies, or one or more symptoms of such disorder or condition. The term “treatment”, as used herein, unless otherwise indicated, refers to the act of treating as “treating” is defined immediately above.

As used herein, the term “patient” includes humans, non-human mammals (e.g., dogs, cats, rabbits, cattle, horses, sheep, goats, swine, deer, and the like) and non-mammals (e.g., birds, and the like). Preferably, the patient is a human patient.

As used herein, the term “advanced” in relation to cancer (e.g. advanced melanoma), unless otherwise indicated, means cancer which is unlikely to be cured or controlled with treatment (that is to say clinically approved treatments). The cancer may have spread from where it first occurred (primary tumour) to other regions of the body, and may therefore be metastatic cancer. Advanced cancer may be referred to as terminal cancer or end-stage cancer.

As used herein, the term “relapsed” in relation to cancer, unless otherwise indicated, means cancer that responded to a previous line of therapy (either partial response [PR], or complete response [CR]) and thus entered a period of remission, but has returned following the conclusion of said previous lines of therapy. Relapsed cancer is sometimes referred to as “recurrent” cancer.

As used herein, the term “refractory” in relation to cancer, unless otherwise indicated, means cancer that did not respond to one or more previous lines of therapy. Refractory cancer is sometimes referred to as being ‘resistant’ to standard of care therapies.

As used herein, the term “adjuvant” in relation to treatment of cancer, unless otherwise indicated, means a treatment administered to a patient in need thereof after administration of a primary treatment (e.g. chemotherapy, radiation therapy, surgery), to reduce the risk of the cancer returning.

As used herein, the term “neoadjuvant” in relation to treatment of cancer, unless otherwise indicated, means a treatment administered to a patient in need thereof prior to administration of a primary treatment (e.g. chemotherapy, radiation therapy, surgery), to reduce the tumour burden prior administration of said primary treatment. For example, where the primary treatment is chemotherapy (e.g. immunotherapy), the neoadjuvant may be administered to induce a biological response (e.g. an immunological response) which increases the efficacy of the primary treatment.

To provide a more concise description, some of the quantitative expressions given herein are not qualified with the term “about”. It is understood that, whether the term “about” is used explicitly or not, every quantity given herein is meant to refer to the actual given value, and it is also meant to refer to the approximation to such given value that would reasonably be inferred based on the ordinary skill in the art, including equivalents and approximations due to the experimental and/or measurement conditions for such given value.

### DETAILED DESCRIPTION

Whilst the following detailed description is described with reference to programmed cell death protein 1 (PD-1) and programmed cell death protein ligand 1 (PD-L1) as exemplary immune checkpoint inhibitors, those skilled in the art will appreciate that the scope of the present invention is not limited to these particular classes of immune checkpoint inhibitor, and that the benefits of the invention may be realised by providing a combination comprising any suitable immune checkpoint inhibitor.

As described above, PD-1 and its interaction with its receptor PD-L1 (and/or PD-L2) have been implicated in promoting tumour cell survival, and therefore represent an attractive target for new immunotherapies. PD-L1 (and PD-L2) have been shown to be upregulated in various cancers, including solid tumours such as melanoma, lung cancer, colorectal cancer, gastric cancer, bladder cancer, pancreatic cancer, prostate cancer ovarian cancer, breast cancer and non-Hodgkin lymphoma (Han Y, Liu D, Li L. PD-1/PD-L1 pathway: current researches in cancer. Am J Cancer Res. 2020 Mar. 1; 10(3):727-742. PMID: 32266087; PMCID: PMC7136921), and immune checkpoint inhibitors against this target have been widely investigated.

Combination therapies comprising one or more immune checkpoint inhibitors have been shown to be more effective in treating cancer than an immune checkpoint inhibitor as a monotherapy. For instance, combination immunotherapies comprising immune checkpoint inhibitors targeting PD-1 and CTLA-4 having been clinically approved (e.g. the anti-PD-1 antibody Nivolumab in combination with the anti-CTLA4 antibody Ipilimumab) for treatment in cancer. For example, a recent clinical review (Johnson et al. Immune-checkpoint inhibitors: long-term implications of toxicity. Nat Rev Clin Oncol 19, 254-267 (2022). https://doi.org/10.1038/s41571-022-00600-w) suggests that in metastatic melanoma, treatment with Nivolumab in combination with Ipilimumab is associated with a 59% response rate (compared with 43% for Nivolumab alone and 15-20% for Ipilimumab alone), and in renal cell carcinoma (RCC) approximately 40% (versus ˜25% with Nivolumab alone and minimal activity for Ipilimumab as a monotherapy). This combination is also approved for use in patients with non-small cell lung cancer (NSCLC), microsatellite instability-high colorectal cancer or hepatocellular carcinoma, in whom CTLA-4 inhibition has minimal (or poorly characterized) activity as a monotherapy.

However, concurrent inhibition of immune checkpoints such as PD-1 and CTLA-4 simultaneously increases the risk of autoimmune toxicities. It has been observed that such combination therapies result in increased incidences of high-grade immune-related adverse events (irAEs). Specifically, Nivolumab monotherapy, Ipilimumab monotherapy and the combination of Ipilimumab plus Nivolumab have been shown to lead to high-grade adverse events in 23%, 28% and 59% of patients with advanced melanoma, respectively (Johnson et al. Immune-checkpoint inhibitors: long-term implications of toxicity. Nat Rev Clin Oncol 19, 254-267 (2022). https://doi.org/10.1038/s41571-022-00600-w).

The present invention addresses this problem in, but not limited to, the provision of a combination according to the first aspect of the present invention, and the method according to the seventh aspect of the present invention. Surprisingly, the combinations and methods disclosed herein not only avoid adverse events in patients associated with existing combinations comprising an immune checkpoint inhibitor, but additionally improves overall survival and progression free survival in a clinical patient population, as described in the examples below in more detail. The present invention therefore represents a promising therapy for use in the treatment of cancer, which reduces or even obviates adverse events in patients receiving the current standard of care comprising combinations of immune checkpoint inhibitors, and moreover improves clinical prognosis.

Moreover, the combinations and methods described herein according to the first aspect of the present invention have been shown to be effective in increasing overall survival, and progression free survival, in patients with advanced disease, that is to say patients who had received one or more previous lines of therapy, and their disease was deemed to be unlikely to be responsive to continued treatment in line with the current standard of care. The examples herein surprisingly describe an observed increase in progression free survival, and overall survival in the order of months, representing a significant delay in the onset of disease progression and death.

In some embodiments, pharmaceutically acceptable salt of tinostamustine according to the compositions and method provided herein is a hydrochloride salt, hydrobromide salt, methanesulfonate, toluenesulfonate, acetate, fumarate, sulfate, bisulfate, succinate, citrate, phosphate, maleate, nitrate, tartrate, benzoate, biocarbonate, carbonate, sodium hydroxide salt, calcium hydroxide salt, potassium hydroxide salt, tromethamine salt, or mixtures thereof.

In some embodiments, the immune checkpoint inhibitor blocks or substantially blocks the binding of PD-L1 to PD-1, for example by the immune checkpoint inhibitor binding to PD-1. In one embodiment, the immune checkpoint inhibitor is any agent or drug that is capable of binding to PD-1 and blocking or substantially blocking the binding of PD-L1 or PD-1. Examples of such agents include peptides, cyclic peptides, small molecules/chemicals, antibodies, functional fragment of an antibody, nanobodies, or aptamers.

In some embodiments, the immune checkpoint inhibitor is an anti PD-1 antibody. Certain embodiments provide that the immune checkpoint inhibitor is selected from Pembrolizumab, Nivolumab, Dostarlimab, Retifanlimab and Cemiplimab.

In some embodiments, the immune checkpoint inhibitor is Pembrolizumab. In such embodiments, the combination or method according to the invention is for use in the treatment of cancer, wherein the cancer is selected from melanoma, non-small cell lung cancer, Hodgkin lymphoma urothelial carcinoma, head and neck squamous cell carcinoma, and renal cell carcinoma.

In some embodiments, the immune checkpoint inhibitor is Nivolumab. In such embodiments, the combination or method according to the invention is for use in the treatment of cancer, wherein the cancer is selected from melanoma, non-small cell lung cancer, malignant pleural mesothelioma, advanced renal cell carcinoma, Hodgkin Lymphoma, squamous cell carcinoma of the head and neck, urothelial carcinoma, microsatellite instability-high or mismatch repair deficient metastatic colorectal cancer, hepatocellular carcinoma, oesophageal cancer, gastric cancer, gastroesophageal junction cancer, and oesophageal adenocarcinoma.

In some embodiments, the immune checkpoint inhibitor is Dostarlimab. In such embodiments, the combination or method according to the invention is for use in the treatment of cancer, wherein the cancer is selected from endometrial cancer, rectal cancer and solid tumours (e.g., sarcomas and carcinomas), optionally advanced solid tumours.

In some embodiments, the immune checkpoint inhibitor is Retifanlimab. In such embodiments, the combination or method according to the invention is for use in the treatment of cancer, wherein the cancer is selected from advanced Merkel cell carcinoma.

In some embodiments, the immune checkpoint inhibitor is Cemiplimab. In such embodiments, the combination or method according to the invention is for use in the treatment of cancer, wherein the cancer is selected from melanoma, metastatic cutaneous squamous cell carcinoma (CSCC), myeloma, lung cancer, and cervical cancer.

In some embodiments, the immune checkpoint inhibitor is a programmed cell death protein ligand 1 (PD-L1) inhibitor and/or programmed cell death protein ligand 2 (PD-L2) inhibitor. In some embodiments, the immune checkpoint inhibitor is a PD-L1 inhibitor.

In some embodiments, the immune checkpoint inhibitor is an anti PD-L1 antibody, including, but not limited to, Atezolizumab, Avelumab, Durvalumab.

In some embodiments, the immune checkpoint inhibitor is Atezolizumab. In such embodiments, the combination or method according to the invention is for use in the treatment of cancer, wherein the cancer is selected from melanoma, urothelial carcinoma, small-cell lung cancer, non-small-cell lung cancer, hepatocellular carcinoma, bladder cancer, renal cell carcinoma and triple-negative breast cancer.

In some embodiments, the immune checkpoint inhibitor is Avelumab. In such embodiments, the combination or method according to the invention is for use in the treatment of cancer, wherein the cancer is selected from melanoma, urothelial carcinoma, Merkel-cell carcinoma, and renal cell carcinoma.

In some embodiments, the immune checkpoint inhibitor is Durvalumab. In such embodiments, the combination or method according to the invention is for use in the treatment of cancer, wherein the cancer is selected from melanoma, urothelial carcinoma, non-small cell lung cancer, small cell lung cancer, non-small cell lung cancer, and biliary tract cancer.

In some embodiments, the combination further comprises one or more further immune checkpoint inhibitors. In some embodiments, the method further comprises administering one or more further immune checkpoint inhibitors.

In some embodiments, the further immune checkpoint inhibitors is a cytotoxic T lymphocyte associated antigen 4 (CTLA-4) inhibitor, optionally wherein the CTLA-4 inhibitor is selected from Ipilimumab and Tremelimumab. One embodiment provides that the CTLA-4 inhibitor is Ipilimumab.

In some embodiments, the further immune checkpoint inhibitor is Ipilimumab. In such embodiments, the combination or method according to the invention is for use in the treatment of cancer, wherein the cancer is selected from melanoma, renal cell carcinoma, colorectal cancer, hepatocellular carcinoma, non-small cell lung cancer, malignant pleural mesothelioma, prostate cancer, and urothelial cancer.

In some embodiments, the further immune checkpoint inhibitor is Tremelimumab. In such embodiments, the combination or method according to the invention is for use in the treatment of cancer, wherein the cancer is selected from melanoma (e.g. advanced melanoma), mesothelioma, and non-small cell lung cancer.

In some embodiments, the combinations according to the present invention further comprises one or more additional pharmaceutically active agents. In some embodiments, the method according to the present invention further comprises administering to the subject one or more additional pharmaceutically active agents. Particularly suitable pharmaceutically active agents are anti-tumor agents having a different mode of action to Tinostamustine and/or the immune checkpoint inhibitor. Suitable anti-tumour agents may include alkylating agents such as nitrosureas, ethylenimines, alkylsulfonates, hydrazines and triazines, and platinum based agents; plant alkaloids, taxanes, vinca alkaloids; anti-tumor antibiotics such as chromomycins, anthracyclines, and miscellaneous antibiotics such as Mitomycin and Bleomycin; anti-metabolites such as folic acid antagonists, pyrimidine antagonists, purine antagonists and adenosine deaminase inhibitors; topoisomerase inhibitors such as topoisomerase I inhibitors, topoisomerase II inhibitors, miscellaneous anti-neoplastics such as ribonucleotide reductase inhibitors, adrenocortical steroid inhibitor, anti-microtubule agents, and retinoids; protein kinases; heat shock proteins, poly-ADP (adenosine diphosphate)-ribose polymerase (PARP), hypoxia-inducible factors (HIF), proteasome, Wnt/Hedgehog/Notch signaling proteins, TNF-alpha, matrix metalloproteinase, farnesyl transferase, apoptosis pathway, histone deacetylases (HDAC), histone acetyltransferases (HAT), and methyltransferase; hormonal therapies, vascular disrupting agent, gene therapy, RNAi cancer therapy, chemoprotective agents, antibody conjugate, cancer immunotherapy such as Interleukin-2, cancer vaccines or monoclonal antibodies; and preferably DNA damaging agents, anti-metabolites, topoisomerase inhibitors, anti-microtubule agents, EGFR inhibitors, HER2 inhibitors, VEGFR2 inhibitors, BRAF inhibitors, Bcr-Abl inhibitors, PDGFR inhibitors, ALK inhibitors, PLK inhibitors, MET inhibitors, epigenetic agents, HSP90 inhibitors, PARP inhibitors, CHK inhibitors, aromatase inhibitor, estrogen receptor antagonist, and antibodies targeting VEGF, HER2, EGFR, CD50, CD20, CD30, CD33, and etc.

In one preferred embodiment of the combination or method of the present invention, the immune checkpoint inhibitor and Tinostamustine are adapted for administration concurrently, sequentially or separately. In one embodiment, the immune checkpoint inhibitor and Tinostamustine are adapted for administration separately.

In some embodiments, Tinostamustine is administered to the patient in a first cycle of treatment without the immune checkpoint inhibitor, and in all subsequent cycles of treatment (e.g. second, third, and so on), Tinostamustine and the immune checkpoint inhibitor are administered to the patient sequentially, concurrently or separately. For example, in some embodiments, Tinostamustine is administered to the patient in a first cycle of treatment without the immune checkpoint inhibitor, then in the second cycle of treatment Tinostamustine and the immune checkpoint inhibitor are administered to the patient sequentially (i.e. one after the other without delay) or separately (i.e. one after a period of time has elapsed following administration of the other), and all subsequent cycles may be in accordance with the second cycle.

In certain embodiments where Tinostamustine and the immune checkpoint inhibitor are administered separately, the immune checkpoint inhibitor is administered to the patient first, followed by Tinostamustine, or a pharmaceutically acceptable salt thereof. In some embodiments, the time between separate administration of the immune checkpoint inhibitor and Tinostamustine, or a pharmaceutically acceptable salt thereof, is from 20 to 120 minutes, from 30 to 90 minutes, or from 50 to 70 minutes (e.g. 60 minutes). In certain embodiments, the time between separate administration of the immune checkpoint inhibitor and Tinostamustine, or a pharmaceutically acceptable salt thereof, is at least 30 minutes.

In some embodiments, the patient is administered the treatment on day 1 of each cycle. In some embodiments, a treatment cycle is at least 7 days, or at least 14 days (e.g. the cycle is 14 days long). In some embodiments, the time between the first cycle of treatment and the second cycle of treatment is at least 7 days, or at least 14 days (e.g. 14 days).

In some preferred embodiments, each cycle of treatment is 14 days long, and on day 1 of cycle 1, Tinostamustine is administered to the patient in need thereof without the immune checkpoint inhibitor. For the second cycle of treatment, on day 1, Tinostamustine and the immune checkpoint inhibitor are administered to the patient in need thereof separately, wherein the immune checkpoint inhibitor is administered first, and Tinostamustine or a pharmaceutically acceptable salt thereof is administered to the patient at least 30 minutes after administration of the immune checkpoint inhibitor. For all subsequent cycles, on day 1 of each cycle, Tinostamustine and the immune checkpoint inhibitor are administered to the patient in need thereof separately, wherein the immune checkpoint inhibitor is administered first, and Tinostamustine or a pharmaceutically acceptable salt thereof is administered to the patient at least 30 minutes after administration of the immune checkpoint inhibitor. The immune checkpoint inhibitor may be a PD-1 inhibitor (e.g. Nivolumab).

In some embodiments of the combination of the present invention, the molar ratio of immune checkpoint inhibitor to Tinostamustine is from 1:8000 to 1:500, from 1:7000 to 1:1000, from 1:6000 to 1:2000, or from 1:5000 to 1:3000 (such as, around 1:4000).

In some embodiments, combinations comprising an immune checkpoint inhibitor and Tinostamustine, or a pharmaceutically acceptable salt thereof, in accordance with the present invention are synergistic combinations. For example, combinations comprising a PD-1 inhibitor and Tinostamustine, or a pharmaceutically acceptable salt thereof, aresynergistic combinations.

In a second aspect, the present invention further provides a pharmaceutical composition comprising a pharmaceutically acceptable diluent or carrier and a combination according to the present invention. Preferred compositions include those comprising the preferred combinations of the present invention as described herein. The pharmaceutically acceptable diluent or carrier of the pharmaceutical composition according to the present invention can be any suitable dispersant, excipient, adjuvant, or other material which acts as a carrier for the active agents of the combination of the present invention and which does not interfere with the active agents present in said combination. Examples of typical pharmaceutically acceptable carriers and diluents may be found in “Remington's Pharmaceutical Sciences” by E. W. Martin and these include water, saline, dextrose solution, serum solution, Ringer's solution, polyethylene glycol (e.g., PEG400), a surfactant (e.g Cremophor), a cyclopolysaccharide (e.g hydroxypropyl-β-cyclodextrin or sulfobutyl ether β-cyclodextrins), a polymer, a liposome, a micelle, a nanosphere, etc.

In some embodiments, a pharmaceutical composition comprising Tinostamustine comprises a cyclodextrin. Cyclodextrins are cyclic oligomers of dextrose with a truncated cone structure consisting of a hydrophilic exterior and a hydrophobic interior cavity. A cyclodextrin can form an inclusion complex with a guest molecule by complexing with all or a portion of a hydrophobic guest molecule within its cavity. The size of the cavity is determined by the number of glucopyranose units in the cyclodextrin. Alpha-(α), beta-(β), and gamma-(γ) cyclodextrins are the most common cyclodextrins and possess six, seven and eight glucopyranose units, respectively. Because natural cyclodextrins have relatively low aqueous solubility and are associated with toxicity, chemically modified cyclodextrin derivatives have been developed to overcome these limitations. Such cyclodextrin derivatives typically possess a chemical modification at one or more of the 2, 3, or 6 position hydroxyl groups. Cyclodextrin derivatives have, for example, been described in U.S. Pat. Nos. 5,134,127; 5,376,645; 5,571,534; 5,874,418; 6,046,177 and 6,133,248, the contents of which are herein incorporated by reference and made a part hereof. As used herein, the terms “cyclodextrin,” “α-cyclodextrin,” β-cyclodextrin and “γ-cyclodextrin” are intended to encompass unmodified cyclodextrins as well as chemically modified derivatives thereof.

In some embodiments, the composition comprises a cyclodextrin selected from the group consisting of α-cyclodextrin, β-cyclodextrin and γ-cyclodextrin. In a certain embodiment, the cyclodextrin is a β-cyclodextrin. In a further embodiment, the cyclodextrin is selected from the group consisting of a hydroxypropyl-β-cyclodextrin (Pitha et al, J Pharm Sci, 84 (8), 927-32 (1995)) and sulfobutyl derivatized-β-cyclodextrin (described, for example, in U.S. Pat. Nos. 5,134,127; 5,376,645; 5,874,418; 6,046,177 and 6,133,248). In another embodiment, the cyclodextrin is a hydroxypropyl β-cyclodextrin. In yet another embodiment, the cyclodextrin is sulfobutylether-β-cyclodextrin. Other preferred cyclopolysaccharides include, but are not limited to, β-cyclodextrin substituted with 2-hydroxy-N,N,N-trimethylpropanammonium, carboxymethylated-β-cyclodextrin, O-phosphated-β-cyclodextrin, succinyl-(2-hydroxyl)propyl-betacyclodextrin, sulfopropylated-β-cyclodextrin, heptakis(6amino-6-deoxy)-β-cyclodextrin, O-sulfated-β-cyclodextrin, and 6-monodeoxy-6-mono(3-hydroxy)propylamino-@-cyclodextrin.

In some embodiments, the cyclodextrin is included in an amount that increases the solubility of the active compound in the composition. In one embodiment, the amount of cyclodextrin included within the composition is the minimal amount needed to solubilize the drug in the composition. In a further embodiment, the composition is a parenteral formulation and the amount of cyclodextrin included within the formulation is the minimal amount of cyclodextrin needed to solubilize the drug.

In some embodiments, the composition comprises at least 2.5%, at least 5%, or at least 10%, (weight/volume) of a cyclodextrin. In some other embodiments, the composition comprises from 2.5 to 40%, from 5% to 20%, from 7.5% to 15% of a cyclodextrin. In yet another embodiment, the composition comprises about 10% of a cyclodextrin.

In some embodiments, the composition further comprises pH adjusting agents. In some further embodiments, the pH adjusting agents are one or more acids, bases, or salts. Examples of acids that may be included in the composition include inorganic acids such as hydrochloric acid, sulfuric acid, phosphoric acid, or mixtures thereof, and organic acids such as citric acid, L(−)-malic acid and L(+)tartaric, acid or mixtures thereof. Examples of bases that may be included in the composition include sodium hydroxide, potassium hydroxide, calcium hydroxide, tromethamine, or mixtures thereof. Examples of salt that may be included in the composition include sodium bicarbonate, sodium carbonate, sodium citrate, or mixtures thereof. In a further embodiment, the composition comprising one or more pH adjusting agents has a pH range of 6.0-9.0, preferably 7.0-8.0.

In some further embodiments, the composition comprises dextran. In yet another embodiment, the composition comprises dextran in an amount of range from about 1% to about 5% weight/volume dextran. In a further embodiment, the composition comprises from about 2 to about 4% weight/volume dextran.

Any inert excipient that is commonly used as a carrier or diluent may be used in compositions of the present invention, such as sugars, polyalcohols, soluble polymers, salts and lipids. Sugars and polyalcohols which may be employed include, without limitation, lactose, sucrose, mannitol, and sorbitol. Illustrative of the soluble polymers which may be employed are polyoxyethylene, poloxamers, polyvinylpyrrolidone, and dextran. Useful salts include, without limitation, sodium chloride, magnesium chloride, and calcium chloride. Lipids which may be employed include, without limitation, fatty acids, glycerol fatty acid esters, glycolipids, and phospholipids.

In addition, the compositions may further comprise binders (e.g., acacia, cornstarch, gelatin, carbomer, ethyl cellulose, guar gum, hydroxypropyl cellulose, hydroxypropyl methyl cellulose, povidone), disintegrating agents (e.g., cornstarch, potato starch, alginic acid, silicon dioxide, croscarmellose sodium, crospovidone, guar gum, sodium starch glycolate, Primogel), buffers (e.g., tris-HCL, acetate, phosphate) of various pH and ionic strength, additives such as albumin or gelatin to prevent absorption to surfaces, detergents (e.g., Tween 20, Tween 80, Pluronic F68, bile acid salts), protease inhibitors, surfactants (e.g., sodium lauryl sulfate), permeation enhancers, solubilizing agents (e.g., glycerol, polyethylene glycerol, cyclodextrins), a glidant (e.g., colloidal silicon dioxide), anti-oxidants (e.g., ascorbic acid, sodium metabisulfite, butylated hydroxyanisole), stabilizers (e.g., hydroxypropyl cellulose, hydroxypropylmethyl cellulose), viscosity increasing agents (e.g., carbomer, colloidal silicon dioxide, ethyl cellulose, guar gum), sweeteners (e.g., sucrose, aspartame, citric acid), flavoring agents (e.g., peppermint, methyl salicylate, or orange flavoring), preservatives (e.g., Thimerosal, benzyl alcohol, parabens), lubricants (e.g., stearic acid, magnesium stearate, polyethylene glycol, sodium lauryl sulfate), flow-aids (e.g., colloidal silicon dioxide), plasticizers (e.g., diethyl phthalate, triethyl citrate), emulsifiers (e.g., carbomer, hydroxypropyl cellulose, sodium lauryl sulfate), polymer coatings (e.g., poloxamers or poloxamines), coating and film forming agents (e.g., ethyl cellulose, acrylates, polymethacrylates) and/or adjuvants.

In some embodiments, the compositions are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

In another aspect, the present invention further provides a kit comprising a combination according to the present invention and, optionally, instructions for treating a patient. Typically, a kit can comprise Tinostamustine, or pharmaceutically acceptable salt thereof, and an immune checkpoint inhibitor together with instructions for treating a patient. Each active agent can be provided in a suitable container. The kit may further comprise a delivery system, e.g. for Tinostamustine or pharmaceutically acceptable salt thereof, or the immune checkpoint inhibitor or any combination thereof.

In some embodiments, the instructions advise administration of Tinostamustine or pharmaceutically acceptable salt thereof, or the immune checkpoint inhibitor of the combination concurrently, sequentially or separately according to variables such as the specific condition being treated, the state of that condition, the activity of the specific compounds employed; the specific combination employed; the age, body weight, general health, sex and diet of the patient; the time of administration, route of administration, and rate of excretion of the specific compounds employed; the duration of the treatment; drugs used in combination or contemporaneously with the specific compounds employed; and like factors well known in the medical arts. Preferred kits according to the present invention include those comprising the preferred combinations of the present invention as described herein.

In another aspect of the present invention, the combination, composition or kit according to the present invention is for use in the treatment of cancer.

In still another aspect of the present invention, Tinostamustine, or a pharmaceutically acceptable salt thereof, is for use in the treatment of cancer, wherein Tinostamustine is used in combination with an immune checkpoint inhibitor.

In yet another aspect of the present invention, an immune checkpoint inhibitor is for use in the treatment of cancer, wherein the immune checkpoint inhibitor is used in combination with Tinostamustine, or a pharmaceutically acceptable salt thereof.

In a further aspect of the present invention, a method of treating cancer in a patient in need thereof comprises administering to said patient the combination, composition or kit according to the present invention. The method comprises administering to a patient in need thereof a therapeutically effective amount of Tinostamustine, or a pharmaceutically acceptable salt thereof, in combination with a therapeutically effective amount of an immune checkpoint inhibitor.

It has been found that the combinations, compositions and kits of the present invention are effective in the treatment of cancer, particularly advanced cancer, and more particularly advanced melanoma.

It has also been found that the combinations, compositions and kits of the present invention are effective in the treatment of colorectal cancer, in particular colorectal carcinoma.

In some embodiments, the cancer is relapsed and/or refractory. In some preferred embodiments, the cancer is metastatic cancer. In some preferred embodiments, the cancer is an unresectable cancer.

Examples of cancers which are treatable by the combinations, compositions and kits of the present invention include hematologic cancers (such as multiple myeloma, lymphoma and leukemia), breast cancer, lung cancer, colorectal cancer, prostate cancer, testicular cancer, pancreatic cancer, liver cancer, stomach cancer, biliary tract cancer, esophageal cancer, gastrointestinal stromal tumor, cervical cancer, ovarian cancer, uterine cancer, renal cancer, melanoma, basal cell carcinoma, squamous cell carcinoma, bladder cancer, sarcoma, mesothelioma, thymoma, myelodysplastic syndrome, brain cancer (e.g. glioblastoma) and myeloproliferative disease.

In some embodiments, the cancer is selected from melanoma, small-cell lung cancer, non-small cell lung cancer, mesothelioma (e.g. malignant pleural mesothelioma), renal cell carcinoma, head and neck cancer, urothelial carcinoma, colorectal cancer, oesophageal squamous cell carcinoma, hepatocellular carcinoma, gastric cancer, oesophageal cancer, oesophageal adenocarcinoma, gastroesophageal junction cancer, Microsatellite Instability-High or Mismatch Repair Deficient Cancer, Primary Mediastinal Large B-Cell Lymphoma (PMBCL), Merkel Cell Carcinoma (MCC), Endometrial Carcinoma, Tumour Mutational Burden-High (TMB-H) Cancer, Cutaneous Squamous Cell Carcinoma (cSCC), soft tissue sarcoma, bone sarcoma, ovarian cancer, triple-negative breast cancer, brain cancer (e.g. glioblastoma), multiple myeloma, Hodgkin lymphoma and non-Hodgkin lymphoma.

In some embodiments, the cancer is selected from melanoma, small-cell lung cancer, non-small cell lung cancer, mesothelioma (e.g. malignant pleural mesothelioma), renal cell carcinoma, head and neck cancer, urothelial carcinoma, colorectal cancer (e.g. MSI-H or dMMR metastatic colorectal cancer), oesophageal squamous cell carcinoma, hepatocellular carcinoma, gastric cancer, oesophageal cancer, oesophageal adenocarcinoma, gastroesophageal junction cancer, Microsatellite Instability-High or Mismatch Repair Deficient Cancer, Primary Mediastinal Large B-Cell Lymphoma (PMBCL), Merkel Cell Carcinoma (MCC), Endometrial Carcinoma, Tumour Mutational Burden-High (TMB-H) Cancer, Cutaneous Squamous Cell Carcinoma (cSCC).

In some embodiments, the cancer is selected from melanoma, soft tissue sarcoma, bone sarcoma, ovarian cancer, triple-negative breast cancer, brain cancer (e.g. glioblastoma), multiple myeloma, Hodgkin lymphoma and non-Hodgkin lymphoma.

In some embodiments, the cancer is selected from a cancer for which an immune checkpoint inhibitor has been clinically approved (e.g. by the U.S. Food and Drug Administration; or the European Medicines Agency) for use in the treatment of said cancer. In some embodiments, the cancer is selected from a cancer for which an PD-1 or PD-L1 inhibitor has been clinically approved for use in the treatment of said cancer. In some embodiments, the cancer is selected from a cancer for which Nivolumab has been clinically approved for use in the treatment of said cancer (e.g. melanoma, non-small cell lung cancer, malignant pleural mesothelioma, renal cell carcinoma, Hodgkin lymphoma, squamous cell carcinoma of the head and neck, urothelial carcinoma, MSI-H or dMMR metastatic colorectal cancer, hepatocellular carcinoma, oesophageal cancer, gastric cancer, and gastroesophageal junction cancer).

Where the combination, composition or kit of the present invention is for use in the treatment of a hematologic cancer, the cancer is preferably selected from multiple myeloma (e.g. active myeloma, plasmacytoma, light chain myeloma or non-secretory myeloma, with all forms being treatable in all phases including relapsed and refractory phases), lymphoma (e.g. Hodgkin lymphoma or non-Hodgkin lymphoma) and leukemia [acute lymphoblastic leukemia (ALL), chronic lymphocytic leukemia (CLL), acute myeloid leukemia (AML, including myeloblastic leukemia, acute promyelocytic leukemia, acute myelomonocytic leukemia, acute monocytic leukemia, acute erythroleukemia and acute megakaryotic leukemia, with all forms being treatable in all phases including relapsed and refractory phases), chronic myeloid leukemia (CML), hairy cell leukemia (HCL), T-cell prolymphocytic leukemia (T-PLL), large granular lymphocytic leukemia or T-cell acute lymphoblastic leukemia.

Where the combination, composition or kit of the present invention is for use in the treatment of brain cancer, optionally wherein the brain cancer is glioma or glioblastoma. Certain embodiments provide that the glioblastoma is selected from a MGMT positive astrocytic brain tumour, a MGMT negative astrocytic brain tumour, a metastatic brain cancer and primary CNS lymphoma. In some embodiments, the glioblastoma is a MGMT positive astrocytic brain tumour. In some embodiments, the glioblastoma is a MGMT negative astrocytic brain tumour.

In particular, the combinations, compositions and kits of the present invention are effective against melanoma, particularly advanced melanoma. Where the combination, composition or kit of the present invention is for use in the treatment of melanoma, the melanoma is preferably selected from acral lentiginous, mucosal, uveal nodular, and superficial spreading melanoma.

In particular, the combinations, compositions and kits of the present invention are effective against colorectal cancer. Where the combination, composition or kit of the present invention is for use in the treatment of colorectal cancer, the colorectal cancer may preferably be selected from a carcinoma, primary colorectal lymphomas, Gastrointestinal stromal tumors, Leiomyosarcomas, carcinoid tumors and melanomas, even more preferably the colorectal cancer is a carcinoma, and preferably the carcinoma is adenocarcinoma.

In accordance with the present invention, Tinostamustine or a pharmaceutically acceptable salt thereof is typically administered to a patient in need thereof at a dosage range of 10 to 50 mg/m² body surface area of the patient, at a dosage range of from 20 to 40 mg/m² body surface area of the patient, at a dosage range of 25 to 40 mg/m² body surface area of the patient, or at a dosage range of from 25 to 35 mg/m² body surface area of the patient (e.g. 30 mg/m²). In some embodiments, Tinostamustine or a pharmaceutically acceptable salt thereof is administered to a patient in need thereof at a dosage range of 10 to 50 mg/kg body weight patient, at a dosage range of 20 to 40 mg/kg body weight patient, at a dosage range of 25 to 40 mg/kg body weight patient, or at a dosage range of 25 to 35 mg/kg body weight patient.

Advantageously, it has been found that administration of Tinostamustine to a patient in need thereof at the dosage ranges indicated above does not adversely affect the immune system, and in particular does not cause significant myelosuppression. In some preferred embodiments, the dosage range of Tinostamustine does not exceed 40 mg/m² body surface area of patient because below 40 mg/m² no myelosuppression is caused. As such, using Tinostamustine, or a pharmaceutically acceptable salt thereof, at the indicated dosage range does not adversely affect the intended mode of action of the immune checkpoint inhibitor comprised in the claimed combination. More particularly, the dosage ranges of Tinostamustine described herein may preferably avoid Tinostamustine causing cytotoxicity to immune cells. For comparison, the doses at which Tinostamustine are administered in accordance with the present invention are below those normally used to achieve an anti-cancer neoplastic effect (e.g. via inducing cytotoxicity in tumour cells) with Tinostamustine as a monotherapy (typically 80-120 mg/kg).

In accordance with the present invention, the immune checkpoint inhibitor is typically administered to the patient in need thereof at a dosage range of 1 to 5 mg/kg body weight of the patient, or at a dosage range of 2 to 4 mg/kg body weight of the patient, or around 3 mg/kg body weight of the patient (e.g. 3 mg/kg). In some embodiments, the immune checkpoint inhibitor is administered to the patient in need thereof at a dosage range of from 1 to 5 mg/m² body surface area of the patient, or at a dosage range of 2 to 4 mg/m² body surface area of the patient, or around 3 mg/m² body surface area of the patient (e.g. 3 mg/m²).

The therapeutically effective amount of a combination, composition or kit according to the present invention is an amount of the combination, composition or kit which confers a therapeutic effect in accordance with the present invention on the treated subject, at a reasonable benefit/risk ratio applicable to any medical treatment. The therapeutic effect may be objective (i.e. measurable by some test or marker) or subjective (i.e. subject gives an indication of or feels an effect). An effective amount of the combination, composition or kit according to the present invention is believed to be one wherein Tinostamustine or a pharmaceutically acceptable salt thereof is included in the combination at a dosage range of from 10 to 50 mg/kg body weight patient (e.g., from 20 to 40 mg/m² body surface area, such as 25, 30, or 35 mg/m² body surface area), and the immune checkpoint inhibitor is included at a dosage range of from 1 to 5 mg/kg body weight patient (e.g., from 2 to 4 mg/kg, such as 3 mg/kg body weight).

Effective doses will vary depending on route of administration, as well as the possibility of co-usage with other active agents. It will be understood, however, that the total daily usage of the combinations, compositions and kits of the present invention will be decided by the attending physician within the scope of sound medical judgment. The specific therapeutically effective dose level for any particular patient will depend upon a variety of factors including the disorder (e.g. cancer) being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or contemporaneously with the specific compound employed; and like factors well known in the medical arts.

In some embodiments, the patient has already received at least one line of therapy, that is to say the patient has already received a course of treatment (e.g. chemotherapy, radiotherapy, surgery) but the cancer is still present in the patient. In some embodiments, the patient has progressive disease and the tumour burden is increasing (e.g. larger tumour volume and/or the cancer is spreading to other areas of the body).

In some embodiments, the patient has received at least one line of previous therapy. In some embodiments, the patient has received at least two lines of previous therapy. In some embodiments, the patient has received at least three lines of previous therapy. In the some embodiments, the patient has received at least four lines of previous therapy. In the some embodiments, the patient has received at least five lines of previous therapy.

In some embodiments, the patient was treated with at least one line of previous therapy which included administration of an immune checkpoint inhibitor. In such embodiments, preferably an immune checkpoint inhibitor has not been administered to the patient for at least six months prior to commencing treatment in accordance with the presently claimed invention. In some embodiments, the patient has not previously been administered with an immune checkpoint inhibitor in a previous line of therapy.

Suitable examples of the administration form of the combination, composition or kit of the present invention include without limitation oral, topical, parenteral, sublingual, rectal, vaginal, ocular, and intranasal. Parenteral administration includes subcutaneous injections, intravenous, intramuscular, intrasternal injection or infusion techniques. In some preferred embodiments, the combinations, compositions and kits are administered parenterally.

Combinations and compositions of the invention can be formulated so as to allow a combination or composition of the present invention to be bioavailable upon administration of the combination or composition to an animal, preferably human. Compositions can take the form of one or more dosage units, where for example, a tablet can be a single dosage unit, and a container of a combination or composition of the present invention in aerosol form can hold a plurality of dosage units.

Preferably the combinations of the present invention are provided in the form of kits. Typically, a kit includes an immune checkpoint inhibitor, and Tinostamustine or a pharmaceutically acceptable salt thereof. In certain embodiments, a kit can include one or more delivery systems, e.g. the immune checkpoint inhibitor, Tinostamustine or a pharmaceutically acceptable salt thereof, and directions for the use of the kit (e.g. instructions for treating a subject). These directions/instructions may advise administering the immune checkpoint inhibitor and Tinostamustine or a pharmaceutically acceptable salt thereof of the combination concurrently, sequentially or separately according to variables such as the specific condition being treated, the state of that condition, the activity of the specific compounds employed; the specific combination employed; the age, body weight, general health, sex and diet of the patient; the time of administration, route of administration, and rate of excretion of the specific compounds employed; the duration of the treatment; drugs used in combination or contemporaneously with the specific compounds employed; and like factors well known in the medical arts.

In some embodiments, the pharmaceutically acceptable diluent or carrier is particulate, so that the compositions are, for example, in tablet or powder form. In some embodiments, the carrier(s) are liquid, with the combinations, compositions or kits being, for example, an oral syrup or injectable liquid. In additional embodiments, the carrier(s) are gaseous, so as to provide an aerosol composition useful in, for example, inhalatory administration. Such pharmaceutical carriers can be liquids, such as water and oils, including those of petroleum, animal, vegetable or synthetic origin, such as peanut oil, soybean oil, mineral oil, sesame oil and the like. The carriers can be saline, gum acacia, gelatin, starch paste, talc, keratin, colloidal silica, urea, and the like. In addition, auxiliary, stabilizing, thickening, lubricating and coloring agents can be used. In one embodiment, when administered to an animal, the combination, composition or kit of the present invention and the pharmaceutically acceptable carriers are sterile. Water is a preferred carrier when the combination or composition of the present invention is administered intravenously. Saline solutions and aqueous dextrose and glycerol solutions can also be employed as liquid carriers, particularly for injectable solutions. Suitable pharmaceutical carriers also include excipients such as starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The present compositions, if desired, can also contain minor amounts of wetting or emulsifying agents, or pH buffering agents.

When intended for oral administration, in some embodiments, the combination, composition or kit is in solid or liquid form, where semi-solid, semi-liquid, suspension and gel forms are included within the forms considered herein as either solid or liquid.

As a solid composition for oral administration, in some embodiments, the combination, composition or kit is formulated into a powder, granule, compressed tablet, pill, capsule, chewing gum, wafer or the like form. Such a solid composition typically contains one or more inert diluents, either as a single tablet comprising all active agents or as a number of separate solid compositions, each comprising a single active agent of the combination of the present invention (in the case of the kit). In addition, one or more of the following can be present: binders such as carboxymethylcellulose, ethyl cellulose, microcrystalline cellulose, or gelatin; excipients such as starch, lactose or dextrins, disintegrating agents such as alginic acid, sodium alginate, corn starch and the like; lubricants such as magnesium stearate; glidants such as colloidal silicon dioxide; sweetening agents such as sucrose or saccharin; a flavoring agent such as peppermint, methyl salicylate or orange flavoring; and a coloring agent.

When the combination or composition is in the form of a capsule (e. g. a gelatin capsule), in some embodiments, it contains, in addition to materials of the above type, a liquid carrier such as polyethylene glycol, cyclodextrin or a fatty oil.

In some embodiments, the combination, composition or kit is in the form of a liquid, e. g. an elixir, syrup, solution, emulsion or suspension. The liquid can be useful for oral administration or for delivery by injection. When intended for oral administration, a combination, composition or kit can comprise one or more of a sweetening agent, preservatives, dye/colorant and flavor enhancer. In a combination or composition for administration by injection, one or more of a surfactant, preservative, wetting agent, dispersing agent, suspending agent, buffer, stabilizer and isotonic agent can also be included. In the kit of the present invention, in some embodiments, the liquid components comprising one or more of the active agents of the composition are combined prior to administration and administered concurrently, or each active agent is administered sequentially or separately.

The preferred route of administration is parenteral administration including, but not limited to, intradermal, intramuscular, intraperitoneal, intravenous, subcutaneous, intranasal, epidural, intranasal, intracerebral, intraventricular, intrathecal, intravaginal or transdermal. The preferred mode of administration is left to the discretion of the practitioner, and will depend in part upon the site of the medical condition (such as the site of cancer).

In one embodiment, the combinations, compositions and kits of the present invention are administered intravenously.

The liquid combinations, compositions and kits of the invention, whether they are solutions, suspensions or other like form, can also include one or more of the following: sterile diluents such as water for injection, saline solution, preferably physiological saline, Ringer's solution, isotonic sodium chloride, fixed oils such as synthetic mono or digylcerides, polyethylene glycols, glycerin, or other solvents; antibacterial agents such as benzyl alcohol or methyl paraben; and agents for the adjustment of tonicity such as sodium chloride or dextrose. A parenteral combination or composition can be enclosed in an ampoule, a disposable syringe or a multiple-dose vial made of glass, plastic or other material. Physiological saline is a preferred adjuvant.

For administration (e.g. intravenous) the combination, in some embodiments, composition or kit typically comprises Tinostamustine or a pharmaceutically acceptable salt thereof at a dosage range of from 10 to 50 mg/m² body surface area of the patient, at a dosage range of 20 to 40 mg/m² body surface area of the patient, at a dosage range of 25 to 40 mg/m² body surface area of the patient, or at a dosage range of 25 to 35 mg/m² body surface area of the patient (e.g. 30 mg/m²), and the immune checkpoint inhibitor at a dosage range of from 1 to 5 mg/m² body surface area of the patient, or at a dosage range of 2 to 4 mg/m² body surface area of the patient, for example, at a dosage range of around 3 mg/m² body surface area of the patient (e.g. 3 mg/m²).

In some embodiments, the combination, composition or kit of the present invention is formulated such that the immune checkpoint inhibitor and Tinostamustine or a pharmaceutically acceptable salt thereof are adapted for administration concurrently, sequentially or separately. In one embodiment, they are administered separately.

The combination, composition or kit of the present invention can be administered by any convenient route, for example by infusion or bolus injection, by absorption through epithelial or mucocutaneous linings.

In specific embodiments, it can be desirable to administer one or more combinations, compositions or kits of the present invention or combinations, compositions or kits locally to the area in need of treatment. In one embodiment, administration can be by direct injection at the site (or former site) of a cancer, tumor or neoplastic or pre-neoplastic tissue.

Pulmonary administration can also be employed, e. g. by use of an inhaler or nebulizer, and formulation with an aerosolizing agent, or via perfusion in a fluorocarbon or synthetic pulmonary surfactant. In certain embodiments, the combination, composition or kit of the present invention or compositions can be formulated as a suppository, with traditional binders and carriers such as triglycerides.

The present combination, composition or kit can take the form of solutions, suspensions, emulsion, tablets, pills, pellets, capsules, capsules containing liquids, powders, sustained-release formulations, suppositories, emulsions, aerosols, sprays, suspensions, or any other form suitable for use. Other examples of suitable pharmaceutical carriers are described in “Remington's Pharmaceutical Sciences” by E. W. Martin.

The pharmaceutical combinations, compositions and kits can be prepared using methodology well known in the pharmaceutical art. For example, a composition intended to be administered by injection can be prepared by combining the components of a kit of the present invention with water so as to form a solution. A surfactant can be added to facilitate the formation of a homogeneous solution or suspension.

The combinations, compositions and kits of the present invention are particularly effective in the treatment of cancer. As described in more detail in the examples below, it has been discovered that the combinations according to the presently claimed invention are more effective in treating cancer than the present current standard of care.

The present disclosure provides use of a combination comprising Tinostamustine, or a pharmaceutically acceptable salt thereof, and an immune checkpoint inhibitor in the treatment of cancer. The cancer and/or said treatment may be as defined in accordance with the aspects of the present invention described above.

The present invention is also directed to the use of a combination, composition or kit according to the present invention in the manufacture of a medicament for the treatment of cancer. The cancer and/or said treatment may be as defined in accordance with any of the aspects of the present invention described above.

Further, the present disclosure provides use of an immune checkpoint inhibitor in the manufacture of a medicament for use in the treatment of cancer, wherein in said treatment the immune checkpoint inhibitor is used in treatment in combination with Tinostamustine, or a pharmaceutically acceptable salt thereof. The cancer and/or said treatment may be as defined in accordance with any of the aspects of the present invention described above.

Still further, the present disclosure provides Tinostamustine, or a pharmaceutically acceptable salt thereof, for use in the treatment of melanoma, such as advanced melanoma. Certain embodiments provide that the melanoma is relapsed and/or refractory. The cancer and/or said treatment may be as defined in accordance with any of the aspects of the present invention described above.

According to a further aspect of the present invention, the present disclosure provides Tinostamustine, or a pharmaceutically acceptable salt thereof, for use in the treatment of colorectal cancer. For example, the colorectal cancer is a carcinoma.

It will be appreciated that the cancer to be treated, and the method of treatment including administering the patient a therapeutically amount of Tinostamustine, or a pharmaceutically acceptable salt thereof, or a therapeutically effective amount of the immune checkpoint inhibitor as defined are equally applicable to all aspects of the present invention as defined herein.

### EXAMPLES

### Example 1

The example below pertains to final clinical results from clinical study registration numbers National Clinical Trials (NCT) 030903458 and Swiss National Clinical Trials Portal (SNCTP) 000003243, which are ongoing.

The clinical study relates to an open-label, single-centre, phase-1B clinical trial to determine the safety, tolerability, recommended dose and anti-cancer efficacy of Tinostamustine in combination with the anti-PD-1 monoclonal antibody (mAb), Nivolumab in patients with advanced melanoma receiving standard-of-care treatment. Tinostamustine is a compound having the following structural formula:

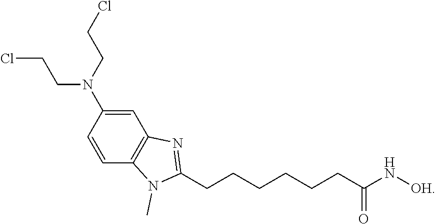 [SMILES: Cn1c(CCCCCCC(=O)NO)nc2cc(N(CCCl)CCCl)ccc21]

Guidance on the current standard of care for patients with metastatic melanoma can be found in Keilholz et al (doi: https://doi.org/10.1016/j.annonc.2020.07.004). As will be appreciated by those skilled in the art, the care given to a particular patient depends on the individual circumstances of the patient.

### Materials

Tinostamustine was provided by Mundipharma Research Limited (Cambridge, U.K.) as a lyophilized powder 100 mg for reconstitution and intravenous administration. Nivolumab was provided by Bristol-Myers-Squibb (BMS) as a concentrate 100 mg/10 mL for (optional dilution and) intravenous administration.

### Key Inclusion Criteria

Patients with either histologically or cytologically confirmed inoperable stage Ill or metastatic stage IV melanoma;
Patients are either (i) not considered for first-line combined anti-PD(L)1/anti-CTLA4 immunotherapy or (ii) have failed standard palliative systemic treatment including anti-PD(L)1/anti-CTLA4 immunotherapy and/or BRAF/MEK-targeting tyrosine kinase inhibitors;
Eastern Cooperation Oncology Group (ECOG) performance status ≤2
Patients with brain metastases must have undergone definitive treatment (surgery or radiotherapy) at least 2 weeks prior to starting study drug and be documented as having stable disease by imaging;
Adequate bone marrow, renal and hepatic function;
Adequate contraception.

### Key Exclusion Criteria

Prior adjuvant treatment with PD(L)1 targeted monoclonal antibody, except patients who completed adjuvant PD(L)1 targeted treatment at least 6 months before start of study treatment;
Patients who discontinued previous adjuvant anti-PD(L)-1 treatment due to anti-PD(L)-1 related toxicities;
Patients who have received systemic treatments or radiotherapy within 2 weeks prior to starting study drug;
Concomitant treatment with systemic steroids at a daily dose equivalent to ≥10 mg of prednisone, or concomitant treatment with immunosuppressive drugs such as methotrexate;
Patients with a prior malignancy (except non-melanoma skin cancers, and in situ cancers such as the following: bladder, colon, cervical/dysplasia, melanoma, or breast). Patients with other second malignancies diagnosed more than 2 years ago who have received therapy with curative intent with no evidence of disease during the interval who are considered by the Investigator to present a low risk for recurrence will be eligible;
New York Heart Association (NYHA) stage Ill/IV congestive heart failure and/or arrhythmia not adequately controlled;
Corrected QT (QTc) interval (Fridericia's formula)>450 msec
Patients who are on treatment with drugs known to prolong the QT/QTc interval (Credible Meds list: Known risk of TdP: https://crediblemeds.org/new-drug-list)
Pregnant and breast feeding patients.

### Patient Cohort

The clinical study recruited patients with advanced melanoma only based on the above exclusion and inclusion criteria. Melanoma has been shown to be responsive to immunotherapy in general, and immune checkpoint inhibitors in particular. The trial therefore focussed on this homogeneous patient population to achieve an insight into clinical activity of the combination of Tinostamustine and an immune checkpoint inhibitor, namely Nivolumab, in patients with advanced melanoma. Those skilled in the art will appreciate, however, that the scope of the present invention is not limited to the treatment of melanoma only, and that the combinations described herein may equally be used in the treatment of any cancer.

A total of 17 patients with advanced melanoma were recruited to a Phase IB study to characterise the safety, tolerability, recommended dose and preliminary efficacy in the treatment of cancer of Tinostamustine in combination with the immune checkpoint inhibitor, Nivolumab.

Thirteen patients (77%) received treatment with an immune checkpoint inhibitor as a previous line of therapy, of which eleven patients (64%) received treatment with an immune checkpoint inhibitor as a first line therapy.

Ten patients (59%) had an elevated lactate dehydrogenase (LDH) baseline. Lactate dehydrogenase (LDH) levels are inversely related with response to checkpoint inhibitors. Elevated LDH levels are the product of enhanced glycolytic activity of the tumor and tumor necrosis due to hypoxia, the latter being associated with high tumor burden (Van Wilpe S, Koornstra R, Den Brok M, De Groot J W, Blank C, De Vries J, Gerritsen W, Mehra N. Lactate dehydrogenase: a marker of diminished antitumor immunity. Oncoimmunology. 2020 Feb. 26; 9(1):1731942. doi: 10.1080/2162402X.2020.1731942. PMID: 32158624; PMCID: PMC7051189).

Four patients (ENI-SG-03, ENISG-01, ENI-SG-10, ENI-SG-15) were treatment naïve, and ten patients had received at least one line of therapy (ENI-SG-13, ENI-SG-06, ENI-SG-16, ENI-SG-05, ENI-SG-14, ENI-GR-04, ENI-SG-11, ENI-SG-12, ENI-SG-08, ENI-GR-17, ENI-SG-09, ENI-SG-07). Seven patients had melanoma phenotypes associated with poor prognosis, including acral lentiginous (3 patients), mucosal (1 patient) and uveal (3 patients). Other melanoma phenotypes included nodular (3 patients) and superficial spreading melanoma (2 patients).

### Dosage

The dose of Tinostamustine is defined herein by reference to amount of Tinostamustine used relative to the patient's surface area, i.e. mg/m². The skilled clinician is able to calculate the patient surface area (also referred to herein as body surface area) using the common general knowledge. In particular, patient surface area (PSA) can be calculated using the following formula (Dubois D, Dubois E F, A formula to estimate the approximate surface area if height and weight be known, Arch Intern Med, 1916, 17, 863-871):

PSA=0.007184×(patient height in cm)^{0.725}×(patient weight in kg)^{0.425}

Tinostamustine was administered intravenously. Four patients were administered tinostamustine at a dose of 15 mg/m² (referred to as dose level 1—DL1) for each cycle. Thirteen patients were administered with Tinostamustine at a dose of 30 mg/m² (referred to as dose level 2—DL2) for each cycle.

Nivolumab was administered intravenously. All patients received Nivolumab in a dose of 3 mg/kg of patient body weight for each cycle, with the exception of cycle 1 (C1) as outlined below. The dose of Nivolumab was calculated dependent on the patient's body weight, and was based on clinical recommendations for use of Nivolumab (NCT01721772; Topalian et al, N Engl J Med 366: 2443-2454, 2012).

Patient body weight and BSA was determined within 2 days prior to each new cycle of treatment.

### Dosage Regimen

Treatment was administered in cycles of 14 days. A total number of 36 cycles was planned as part of the study.

First Cycle (C1)—on the first day (D1) of the first cycle (C1) only Tinostamustine was administered to the patient at the indicated dose, and no Nivolumab was administered to the patient. Tinostamustine was administered intravenously via infusion for one hour.

Second Cycle (C2)—on the first day (D1) of the second cycle (C2), Nivolumab was administered to the patient at 3 mg/kg intravenously via infusion for one hour. Tinostamustine was administered to the patient at least 30 minutes after administration of Nivolumab was completed, at the indicated dose. Tinostamustine was administered intravenously via infusion for one hour.

All Subsequent Cycles—Following C2, on the first day (D1) of each subsequent cycle (Cn; where n is selected from consecutive integers from 3 to 36 inclusive, depending on the total number of cycles the patient participated in the study), Nivolumab was administered to the patient at 3 mg/kg intravenously via infusion for 30 minutes. Tinostamustine was administered to the patient at least 30 minutes after administration of Nivolumab was completed, at the indicated dose. Tinostamustine was administered intravenously via infusion for one-hour.

As such, Nivolumab and Tinostamustine were administered to the patient every two weeks (i.e. on day 1 of each cycle beginning with cycle 2).

### Endpoints

Treatment continued until whichever of the following occurred first: progressive disease, inacceptable toxicity, withdrawal of informed consent or at the investigator's discretion. Patients with progressive disease could continue with the study, provided there was clinical benefit in continuing.

The primary endpoint of the study was dose limiting toxicity (DLT) observed during the first 6 weeks of trial treatment.

The secondary endpoints of the study included:
Objective tumour response (OR) to study treatment according to Response Evaluation Criteria in Solid Tumors (RECIST) v1.1 and iRECIST;
All adverse events (AE) according to common terminology criteria for adverse events (CTCAE);
Progression-free survival (PFS), defined as the time between registration to the study and the time of disease progression or death of the patient, whatever occurs first
Overall survival (OS)

Objective tumour response (OR) can be defined as complete response (CR) or partial response (PR) during trial treatment. Progression free survival (PFS) can be defined as the time between registration to the study and the time of disease progression (progressive disease; PD) or death of the patient, whichever occurs first. Overall survival (OS) is defined as the length of time from the start of the study treatment until death for any reason.

Patients were followed up until the later of at least 100 days after last study treatment with Nivolumab, or at least 30 days after last study treatment with Tinostamustine. Patients were followed up to assess disease progression and overall survival.

### Results

The following data reports the number of cycles of treatment received, as well as days of PFS and OS for each recruited patient.

TABLE 1
Patients were recruited to the clinical study and administered Tinostamustine
in combination with Nivolumab in accordance with the exemplary dosage
regimen described above. For each patient, the number of cycles the
patient remained enrolled in the study is indicated, together with
the number of days and months the patient was determined to have progression
free survival (PFS), and the number of days and months the patient
was determined to have overall survival (PFS).

| | Tinostamustine | | Days | Months^{a} | Days | Months^{b} |
|---|---|---|---|---|---|---|
| Patient I.D. | Dose | # Cycles | PFS | PFS | OS | OS |
| ENI-SG-01 | 15 mg/m2 | 17 | 161 | 5.29 | 300 | 9.87 |
| ENI-SG-02^{c} | 15 mg/m2 | 2 | 17 | 0.56 | 34 | 1.12 |
| ENI-SG-03 | 15 mg/m2 | 36 | 499 | 16.41 | 1168 | 38.41 |
| ENI-GR-04 | 15 mg/m2 | 10 | 623 | 20.49 | 1198^{d} | 39.39 |
| ENI-SG-05^{c} | 30 mg/m2 | 12 | 57 | 1.87 | 435 | 14.30 |
| ENI-SG-06^{c} | 30 mg/m2 | 16 | 240 | 7.89 | 355^{e} | 11.67 |
| ENI-SG-07^{c} | 30 mg/m2 | 1 | 14 | 0.46 | 14 | 0.46 |
| ENI-SG-08^{c} | 30 mg/m2 | 4 | 47 | 1.55 | 60 | 1.97 |
| ENI-SG-09^{c} | 30 mg/m2 | 1 | 16 | 0.53 | 16 | 0.53 |
| ENI-SG-10 | 30 mg/m2 | 8 | 59 | 1.94 | 288 | 9.47 |
| ENI-SG-11^{c} | 30 mg/m2 | 6 | 59 | 1.94 | 128 | 4.21 |
| ENI-SG-12^{c} | 30 mg/m2 | 4 | 50 | 1.64 | 64 | 2.10 |
| ENI-SG-13^{c} | 30 mg/m2 | 24 | 333 | 10.95 | 811 | 26.67 |
| ENI-SG-14^{c} | 30 mg/m2 | 11 | 50 | 1.64 | 174 | 5.72 |
| ENI-SG-15 | 30 mg/m2 | 5 | 64 | 2.10 | 165 | 5.43 |
| ENI-SG-16 | 30 mg/m2 | 15 | 454^{e} | 14.93 | 681^{e} | 22.39 |
| ENI-GR-17^{c} | 30 mg/m2 | 4 | 52 | 1.71 | 681^{d} | 22.39 |

^{a}calculated as [days to PFS/(365/12)];
^{b}calculated as [days OS/(365/12)];
^{c}Patient received a checkpoint inhibitor as a first line therapy;
^{d}ENI-GR-04, and ENI-GR-17 patients completed study alive.;
^{e}Patient ENI-SG-06 was lost to follow up.

Three patients withdrew from the study due to progressive disease at an early stage of the study. As outlined in more detail below, fourteen patients continued with the study to form the study cohort.

The safety part of the study proceeded without any without any significant safety concerns identified for administration of Tinostamustine at doses between 10 mg/m² body surface area and 30 mg/m² body surface area. As such, the recommended dose of Tinostamustine was determined to be 30 mg/m².

For the efficacy part of the study, the mean number of cycles for all patients was 10.8 (22 weeks). For all patients, the median progression free survival (mPFS) was 1.94 (95% CI 1.55, 7.89) months. For all patients, the median overall survival (mOS) was 9.27 (95% CI 1.97, 22.29) months. One study treatment-associated serious adverse events (SAE) was observed, namely Nivolumab-associated immune-related pneumonitis (ENI-GR-04). No other patients were determined to have treatment-related adverse events.

Three patients (ENI-SG-02, ENI-SG-07, ENI-SG-09) received less than or equal to 4 weeks (i.e. less than or equal to 2 cycles) of study treatment due to disease progression at an early stage of the study.

Of the remaining fourteen patients, stable disease was observed in three patients (21.4%; ENI-SG-01, ENI-GR-04, ENI-SG-06). A partial response was observed in three patients (21.4%; ENI-SG-03; ENI-SG-13; ENI-SG-16).

As such, seven patients (ENI-SG-1, ENI-SG-3, ENI-GR-4, ENI-SG-6, ENI-SG-13, ENI-SG-15, and ENI-SG-16; shaded in Table 1 above) were deemed to have a clinical response to treatment, defined by at least 60 days without disease progression (PFS=≥60 days). Taking only these patients who were deemed to have a clinical response to therapy into account, median progression free survival (mPFS) increased to 10.95 (Cl 95% 2.10, 16.41) months, and median overall survival (mOS) increased to 26.67 (5.43, NE) months.

Two patients (ENI-GR-04, and ENI-SG-16) were alive at the time of completion study.

The mPFS and mOS data outlined above was compared with previously published reference data which describe mPFS and mOS for the current standard of care for advanced melanoma, namely a combination of Nivolumab and Ipilimumab. The references outlined below and mPFS and mOS are considered by those skilled in the art to be representative of expected clinical outcome for patients with advanced melanoma treated with a combination of Nivolumab and Ipilimumab as a second line therapy.

One study reports mPFS for treatment of patients with advanced melanoma with a combination of Nivolumab and Ipilimumab as a second line therapy as 2 months (Zimmer L et al; Ipilimumab alone or in combination with nivolumab after progression on anti-PD-1 therapy in advanced melanoma. Eur J Cancer. 2017 April; 75:47-55. doi: 10.1016/j.ejca.2017.01.009. Epub 2017 Feb. 17. PMID: 28214657).

Another study reports mOS for treatment of patients with advanced melanoma with a combination of Nivolumab and Ipilimumab as a second line therapy as 5.6 months (Baron K et al; Comparative effectiveness of second-line ipilimumab vs. nivolumab in combination with ipilimumab in patients with advanced melanoma who received frontline anti-PD-1 antibodies. Journal of Oncology Pharmacy Practice. 2021; 27(3):555-559. doi:10.1177/1078155220924719).

The median progression free survival and median overall survival of patients according to the studies outlined above can be summarised as follows below (Table 2):

TABLE 2
Summary of median progression free survival (mPFS)
and overall survival (mOS) for patients receiving
a second line therapy with advanced melanoma.

| 2^{nd} Line Therapy in patients | | | |
|---|---|---|---|
| with advanced melanoma | mPFS | mos | Reference |
| Tinostamustine + | 1.94 | 9.27 | |
| Nivolumab | (*10.95) | (*26.67) | |
| Nivolumab + Ipilimumab | 2 | | Zimmer et al |
| | | 5.6 | Baron et al |

(*)= data for the ten patients deemed to respond to therapy in accordance with the present examples as outlined above.

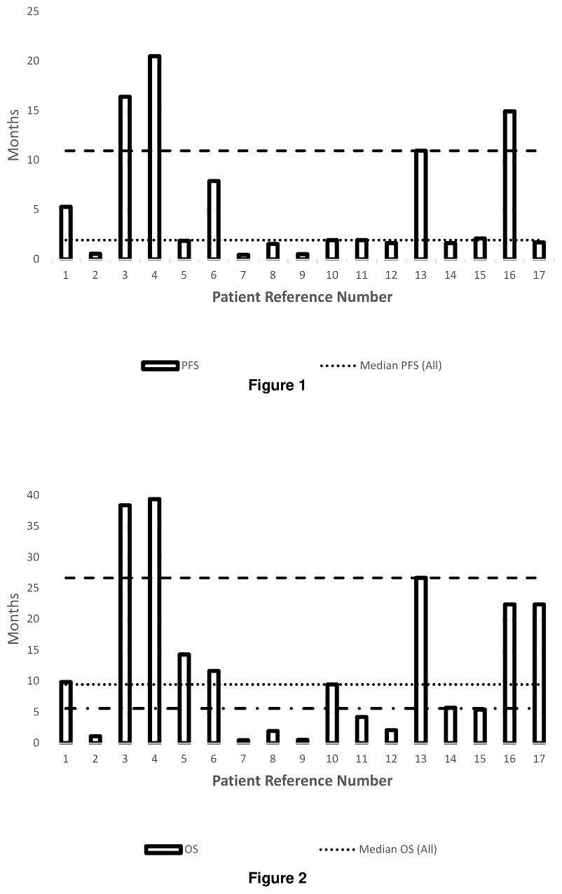

FIG. 1 illustrates PFS for all patients recruited in the study (see Table 1 above), and further includes a comparison of mPFS for all patients, mPFS for patients deemed to respond to treatment (PFS=≥60 days), and mPFS reported by Zimmer et al (reference provided above; see Table 2). These data suggest that a combination of Nivolumab and Tinostamustine compared to a combination of Nivolumab and Ipilimumab which is the current gold standard of care, rising to an increase in mPFS of 8.95 months for patients deemed to respond to treatment (PFS=≥60 days).

FIG. 2 illustrates OS for all patients recruited in the study (see Table 1 above), and further includes a comparison of mOS for all patients, mOS for patients deemed to respond to treatment (PFS=≥60 days), and mOS reported by Baron et al (reference provided above). These data suggest that a combination of Nivolumab and Tinostamustine increases mOS by 3.87 months compared to a combination of Nivolumab and Ipilimumab which is the current gold standard of care, rising to an increase in mOS of 21.07 months for patients deemed to respond to treatment (PFS=≥60 days).

These data demonstrate for the first time that a combination of Tinostamustine and Nivolumab is an effective treatment in the treatment of cancer, particularly in the treatment of advanced melanoma. It has been surprisingly discovered that the combination of Tinostamustine and Nivolumab has been shown to increase PFS and OS compared to the current standard of care, and therefore represents a promising therapy for use in the treatment of cancer. Those skilled in the art will appreciate that Nivolumab is approved for use in the treatment of several forms of cancer, and so the surprisingly effective combination of Tinostamustine and Nivolumab as described herein for the first time may be used in the treatment of any cancer.

### Example 2

We tested the in vivo efficacy of Tinostamustine and an immune checkpoint inhibitor in the treatment of the colorectal murine cancer and has been found to be effective. Specifically, the effects of the combined therapy Tinostamustine and an anti-PLD1 antibody in the treatment of the MC38 subcutaneous murine colorectal syngeneic model in female C57BL/6 mice following 3 dose cycles was assessed. MC38 is a commonly used murine model for colorectal carcinoma.

Six groups of mice were randomly allocated a group, hereafter referred to as Groups 1 to 6, as in Table 3.

TABLE 3
Treatment regime of groups of mice according to Example 2.

| | | | | | Dosing |
|---|---|---|---|---|---|
| | | | | | Frequency |
| | No. of | | Dose level | Method of | and |
| Group | mice | Treatment | (mg/kg) | admin. | Duration |
| 1 | 15 | Vehicle control | 0 | i.v. | HPBCD |
| | | | | | QW × 3 |
| | | | | | weeks |
| 2 | 15 | Anti-PD1 | 10 | i.p. | BIW × 3 |
| | | | | | weeks |
| 3 | 15 | Tinostamustine | 5 | i.v. | QW × 3 |
| | | | | | weeks |
| 4 | 15 | Tinostamustine | 15 | i.v. | QW × 3 |
| | | | | | weeks |
| 5 | 15 | Tinostamustine | 5 | i.v. | QW × 3 |
| | | | | | weeks |
| | 15 | Anti-PD1 | 10 | i.p. | BIW × 3 |
| | | | | | weeks |
| 6 | 15 | Tinostamustine | 15 | i.v. | QW × 3 |
| | | | | | weeks |
| | 15 | Anti-PD1 | 10 | i,p. | BIW × 3 |
| | | | | | weeks |

ROA: i.p. = Intraperitoneal/i.v. = intravenous;
Dosing Frequeny: QW = once weekly;
BIW = Bi-weekly

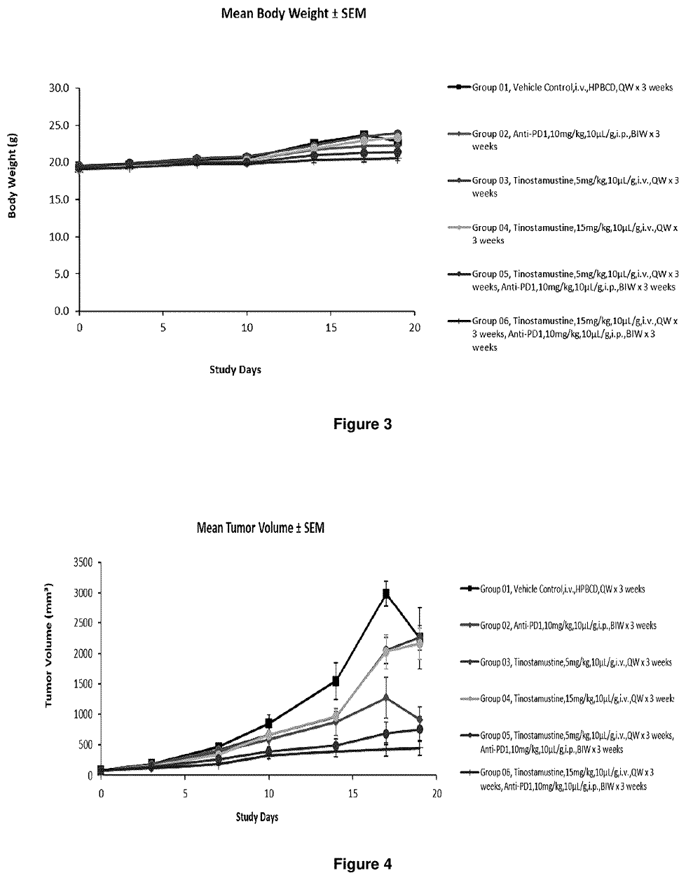

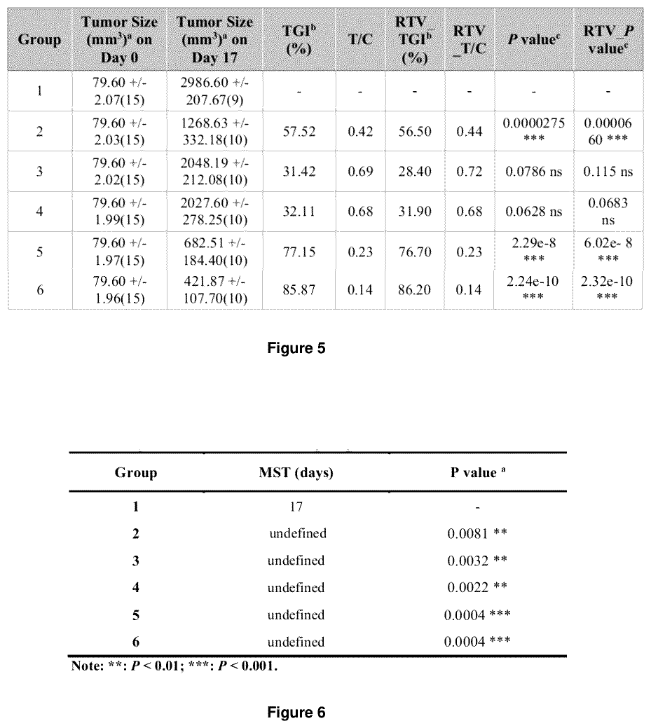

We have shown in FIGS. 3 to 6 that a treatment regime of Tinostamustine and an immune checkpoint inhibitor is effective against colorectal cancer in vivo. Specifically, a treatment regime of Tinostamustine and an PD-1 inhibitor (Anti-mouse PD1, RMP1-14, Bioxcell) was effective in treating a B16-F10 Subcutaneous Murine Melanoma Syngeneic Model in Female C57BL/6 Mice.

FIG. 3 shows that monotherapy of Tinostamustine or a combined therapy with an anti-PD1 inhibitor was well-tolerated by mice, and resulted in no loss of body weight. In fact, in both monotherapy and combination therapy groups, the mean body weight of mice during treatment. Treatment with a combined therapy (Group 5 and 6) achieved a significant reduction in tumour burden to the control (Group 1) and also groups treated with a monotherapy of Tinostamustine.

In support of this, FIG. 6 shows that the claimed combined therapy significantly increased the mean survival of mice compared to the vehicle control, and also the monotherapy with Tinostamustine.

In summary, compared with the vehicle group, the combination treatment (Group 5: Tinostamustine 5 mg/kg and Anti-PD1 10 mg/kg, Group 6: Tinostamustine 15 mg/kg and Anti-PD1 10 mg/kg) showed significant anti-tumour efficacy. Furthermore, the combination treatment groups showed an additive effect in MC38 model. The single treatment of Tinostamustine and together with an Anti-PD1 inhibitor significantly prolonged the survival of the mice in MC38 model. In this study, the mice tolerated the agents well, and there was no significant body weight loss during the study.

### Methods and Materials

### Dosage

According to Example 2, the dosage of the combination therapy may be 5 mg/kg or 15 mg/kg of tinostamustine and 10 mg/kg for a PD-1 inhibitor. According to Example 2, the dosing regime is preferably once weekly for tinostamustine and bi-weekly for a PD-1 inhibitor.

Tinostamustine was administered intravenously, specifically by slow intravenous injection at a rate of ˜60 s per mouse. Two groups of mice were administered tinostamustine at a dose of 5 mg/kg once weekly, either as a monotherapy (Group 3) or as a combination therapy (Group 5). Two groups of mice were administered tinostamustine at a dose of 15 mg/kg once weekly, either as a monotherapy (Group 4) or as a combination therapy (Group 6).

An anti-PDL1 antibody (RMP1-14) was administered by intraperitoneal injection into combination therapy groups (Group 4 and 6). Mice of Groups 4 and 6 received anti-PDL1 antibody in a dose of 10 mg/kg of mouse body weight administered per bi-weekly.

### Cell Culture

The MC38 cells were maintained in vitro with DMEM medium supplemented with 10% fetal bovine serum at 37° C. in an atmosphere of 5% CO2 in air. The cells in exponential growth phase were harvested and quantitated by cell counter before tumour inoculation.

### Tumour Inoculation

Each mouse was inoculated subcutaneously in the right lower flank region with MC38 tumour cells (1×106) in 0.1 ml of PBS for tumour development.

### Randomization

The randomization started when the mean tumour size reached approximately 79 mm3. 90 mice were allocated on to the study and all animals were randomly allocated to 6 study groups, 15 mice in each group.

### Test Article Administration

The treatment was initiated on day 0 per study design.

### Observation and Data Collection

After tumour cell inoculation, the animals were checked daily for morbidity and mortality. During routine monitoring, the animals were checked for treatment effects on tumour growth, behaviour such as mobility, food and water consumption, body weight gain/loss (Body weights) were measured twice a week after randomization), eye/hair matting and any other abnormalities. Mortality and observed clinical signs were recorded for individual animals in detail.

Tumour volumes were measured twice a week after randomization in two dimensions using a caliper, and the volume was expressed in mm3 using the formula: “V=(L×W×W)/2, where V was tumour volume, L was tumour length (the longest tumor dimension) and W was tumour width (the longest tumour dimension perpendicular to L). Dosing as well as tumour and body weight measurements were conducted in a Laminar Flow Cabinet. The body weights and tumour volumes were measured by using StudyDirector™ software (version 3.1.399.19).

### Study Termination

The treatments were performed for 20 days (Day0-Day19). The study was terminated on day 19.

1. A method of treating cancer, the method comprising administering to a subject in need thereof a therapeutically effective amount of Tinostamustine, or a pharmaceutically acceptable salt thereof, in combination with a therapeutically effective amount of an immune checkpoint inhibitor.

2. The method according to claim 1, wherein the immune checkpoint inhibitor is a programmed cell death protein-1 (PD-1) inhibitor.

3. The method according to claim 2, wherein the immune checkpoint inhibitor is an anti PD-1 antibody.

4. The method according to claim 3, wherein the immune checkpoint inhibitor is selected from Pembrolizumab, Nivolumab, Dostarlimab, Retifanlimab and Cemiplimab.

5. The method according to claim 1, wherein the immune checkpoint inhibitor is a programmed cell death protein ligand 1 (PD-L1) inhibitor and/or programmed cell death protein ligand 2 (PD-L2) inhibitor.

6. The method according to claim 5, wherein the immune checkpoint inhibitor is a PD-L1 inhibitor that is an anti PD-L1 antibody.

7. The method according to claim 5 where in the anti PD-L1 antibody is Atezolizumab, Avelumab, or Durvalumab

8. The method according to claim 1, further comprising administering a cytotoxic T lymphocyte associated antigen 4 (CTLA-4) inhibitor.

9. The method of claim 8, wherein the CTLA-4 inhibitor is Ipilimumab or Tremelimumab.

10. The method according to claim 1, wherein the cancer is selected from the group consisting of melanoma, small-cell lung cancer, non-small cell lung cancer, mesothelioma (e.g. malignant pleural mesothelioma), renal cell carcinoma, head and neck cancer, urothelial carcinoma, colorectal cancer, oesophageal squamous cell carcinoma, hepatocellular carcinoma, gastric cancer, oesophageal cancer, oesophageal adenocarcinoma, gastroesophageal junction cancer, Microsatellite Instability-High or Mismatch Repair Deficient Cancer, Primary Mediastinal Large B-Cell Lymphoma (PMBCL), Merkel Cell Carcinoma (MCC), Endometrial Carcinoma, Tumour Mutational Burden-High (TMB-H) Cancer, Cutaneous Squamous Cell Carcinoma (cSCC), soft tissue sarcoma, bone sarcoma, ovarian cancer, triple-negative breast cancer, glioblastoma, multiple myeloma, Hodgkin lymphoma and non-Hodgkin lymphoma.

11. (canceled)

12. The method of claim 10, wherein the cancer is advanced melanoma.

13. (canceled)

14. The method according to claim 1, wherein the cancer is relapsed and/or refractory.

15. The method according to claim 1, wherein in said treatment, Tinostamustine is administered to a patient in need thereof as an adjuvant and/or neoadjuvant.

16. The method according to claim 1, wherein in said treatment, Tinostamustine, or a pharmaceutically acceptable salt thereof, is administered to the patient at a dosage range of from 10 to 50 mg/m² body surface area.

17. The method according to claim 1, wherein in said treatment, Tinostamustine, or a pharmaceutically acceptable salt thereof, and the immune checkpoint inhibitor, are administered to the patient sequentially, concurrently or separately.

18. The method according to claim 17, wherein Tinostamustine, or a pharmaceutically acceptable salt thereof, and the immune checkpoint inhibitor are administered separately.

19. The method according to claim 18, wherein Tinostamustine is administered to the patient in need thereof from 20 to 120 minutes.

20. The method according to claim 1, wherein for a first cycle of treatment Tinostamustine, or a pharmaceutically acceptable salt thereof, is administered to the patient in need thereof without the checkpoint inhibitor, and for all subsequent cycles, Tinostamustine and or a pharmaceutically acceptable salt thereof, and the immune checkpoint inhibitor, are administered to the patient sequentially, concurrently or separately, preferably separately.

21. A method of treating melanoma, comprising administering to a subject in need thereof a therapeutically effective amount of Tinostamustine, or a pharmaceutically acceptable salt thereof.

22. A method of treating colorectal cancer, comprising administering to a subject in need thereof a therapeutically effective amount of Tinostamustine, or a pharmaceutically acceptable salt thereof.
